SEQUENCE LISTING (74 sequences)

SEQ ID NO: 1 - DNA, 1429 bp, Methylomonas sp. 16a
cggtatgcttaacacatgcaagtcgaacgctgaagggtgcttgcacctggatgagtggcggacgggtgagtaatgcataggaatctgcctattagtgggggataacgtggggaaactcacgctaataccgcatacgctctacggaggaaagccggggaccttcgggcctggcgctaatagatgagcctatgtcggattagctagttggtggggtaaaggcctaccaaggcgacgatccgtagctggtctgagaggatgatcagccacactgggactgagacacggcccagactcctacgggaggcagcagtggggaatattggacaatgggcgcaagcctgatccagcaataccgcgtgtgtgaagaaggcctgagggttgtaaagcactttcaatgggaaggaacacctatcggttaatacccggtagactgacattacccatacaagaagcaccggctaactccgtgccagcagccgcggtaatacggagggtgcaagcgttaatcggaattactgggcgtaaagcgtgcgtaggcggttttttaagtcagatgtgaaagccctgggcttaacctgggaactgcatttgatactggggaactagagttgagtagaggagagtggaatttcaggtgtagcggtgaaatgcgtagagatctgaaggaacaccagtggcgaaggcggctctctggactcaaactgacgctgaggtacgaaagcgtgggtagcaaacaggattagataccctggtagtccacgccgtaaacgatgtcaactaaccgttgggttcttaaagaacttagtggtggagctaacgtattaagttgaccgcctggggagtacggccgcaaggctaaaactcaaatgaattgacgggggcccgcacaagcggtggagcatgtggtttaattcgatgcaacgcgaagaaccttacctacccttgacatcctcggaacttgtcagagatgacttggtgccttcgggaaccgagagacaggtgctgcatggctgtcgtcagctcgtgtcgtgagatgttgggttaagtcccgtaacgagcgcaacccttatccttagttgccagcgcgtcatggcgggaactctagggagactgccggtgataaaccggaggaaggtggggacgacgtcaagtcatcatggcccttatgggtagggctacacacgtgctacaatggtcggtacagagggttgcgaactcgcgagagccagccaatcccaaaaagccgatcctagtccggattgcagtctgcaactcgacttgcatgaagtcggaatcgctagtaatcgcggatcagaatgccgcggtgaatacgttcccgggccttgtacacaccgcccgtcacaccatgggagtgggttgcaaaagaagtaggtagtttaaccttcgggagggcgcttaccactttgtg

SEQ ID NO: 2 - protein, 544 aa, artificial seuqence
  constructed variant of P alba IspS
MEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFCFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 3 - DNA, 1632 bp, artificial sequence
  IspS MEA S288C coding opt for E. coli
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgc

SEQ ID NO: 4 - DNA, 1632 bp, artificial sequence
  constructed coding seq for IspS MEA S288S codon optimized for methylomonas
atggaggcccgcagaagcgccaactatgagccgaatagctgggactacgactacctgttatcgtccgataccgatgagtccatcgaggtctacaaagacaaagccaaaaagctggaggccgaggttcgccgtgagatcaacaatgagaaggcagagttcttgaccttgctagagttgatagataacgtgcaaaggctcggattgggttatcggttcgagtcagatatccgaggagcactggaccgctttgtctcgtctggaggtttcgatgcagtgaccaaaacgtcgttgcatggcaccgctttgagtttccgtctgttgcgtcaacatggattcgaggtttcacaagaggcgttctccggcttcaaagatcaaaacggtaacttcctggagaacctgaaagaggacatcaaagccattctgagcttgtatgaggcgagctttttggcgctggagggagagaatatcctggatgaggcgaaagtctttgcgatcagtcatctaaaggagttgtcggaggagaaaatcggcaaagagttggcggagcaggtgaaccacgcgttggagctgccgttacaccgcagaacccaacgcctagaggccgtttggtccattgaggcttaccggaaaaaggaggatgccaatcaagtgctgttagagctggccatactggactacaacatgattcagagcgtgtaccaacgcgacttgcgcgagacaagccgctggtggcgtcgagtcggactggccaccaaactgcactttgcccgtgaccgcctgattgagtccttttactgggcagttggcgtcgcgttcgagccccagtacagcgactgccggaatagcgttgcgaaaatgttctgctttgtaaccatcattgacgatatctatgatgtctatggcaccttggatgagttggagctcttcactgacgccgtcgagcgttgggatgtgaatgccatcaatgaccttccagactatatgaagctgtgttttctcgccttgtataacaccatcaacgagatcgcctacgacaacctgaaggataagggcgagaatatcttgccgtacttgaccaaagcttgggctgatctgtgtaacgccttcttgcaggaggcgaaatggttgtataacaaaagtacgcctactttcgacgattacttcggcaacgcatggaaatcgtcatctggaccgctgcaattggtctttgcgtatttcgccgtggtacaaaacatcaaaaaggaggagatagagaatttgcagaagtatcatgacacgatctccaggccatcccatatcttccgcctctgcaatgatcttgcttcggcgagtgccgagattgcccgtggagagaccgccaattctgtgtcatgctatatgcgcaccaagggaatcagcgaggagctggcgaccgagtcggtaatgaaccttattgacgagacctggaaaaagatgaacaaagagaagttgggtgggtcgctgttcgccaaaccctttgtcgagacggctatcaatctggccagacagagccactgcacttaccataacggcgacgcgcacacctcccctgacgagctgacacggaaacgcgtgctgtcggtcattacggagcccatcttaccgtttgagcgc

SEQ ID NO: 5 - DNA, 198 bp, artificial sequence
  Pcat promoter from plasmid pC194
cctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaa

SEQ ID NO: 6 - protein, 288 aa, Sacharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 7 - DNA, 864 bp, Saccharomyces cerevisiae
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgcta

SEQ ID NO: 8 - DNA, 864 bp, artificial sequence
  yeast IDI encoding sequence codon optimized for expression in methylomonas
atgacagccgacaacaatagcatgccgcatggagccgtcagctcctatgcgaaactggtccaaaatcaaaccccagaggatatcttggaggagttcccggagatcattccgttacaacagcgtccaaacacccggtcgtctgagacgagcaacgacgagtcaggcgagacatgcttcagcggacatgacgaggagcaaatcaaattgatgaacgagaactgcatcgttttagactgggacgataatgcgataggagcgggaaccaaaaaggtttgtcacttgatggagaacattgagaaaggcctgttgcatagggccttcagtgtgttcatctttaacgagcagggcgagctgcttctacaacagcgagccaccgagaagatcaccttcccggacctgtggaccaatacctgctgttcccatccactgtgcatcgacgatgagctgggcctgaaaggaaaactggacgataagatcaagggtgccattactgccgctgtacgtaaactggaccatgagttgggcatcccggaggatgagacgaaaactcgcggaaagtttcacttcttgaatcgcatccactatatggctcccagtaacgagccctggggtgagcacgagattgattacattctgttttacaaaatcaacgcgaaggagaatttgaccgtgaaccccaatgtgaacgaggtccgcgatttcaaatgggtgtcccctaatgatttgaaaacgatgttcgcagatccgtcgtataagtttaccccttggttcaaaatcatttgcgagaactacctgtttaactggtgggagcaattggacgatttgtcggaggtcgagaatgaccgccagatacacagaatgctc

SEQ ID NO: 9 - protein, 344 aa, Pantoea stewartii
MKDTDLTKRKNDHLDIVLRNTAPASGSFARWHFTHCALPELHLDQIDLRTRLFDRPMQAPFLISSMTGGAARALSINHHLAEAAQTLGLALGVGSQRVALESDNDSGLTRDLRRIAPDIPLLANLGAAQILGEQGRRLARNAVSMIEADALIVHLNPLQEALQRGGDRDWRGVLQAIAQLVKSLEVPVVVKEVGAGISAEVAQRLAEAGVSMIDIAGAGGTSWAAVEGERASTPQQRAVAMAFASWGIPTDEALRAVRDRLPAIPLIASGGIRDGIDAAKALRLGADIVGQAAAVLSSALHSTDAVVAHFNTLIEQLRVACFCTGSANLRQLRLAPLHRAGETL

SEQ ID NO: 10 - DNA, 1035 bp, Pantoea stewartii
atgaaggacacggacctgacgaagcgcaaaaacgatcatctggacattgttctgcgtaataccgcgccggcgtcgggcagcttcgcccgctggcactttacccactgcgccctgccggagctgcacctggatcagatcgatctgcgcacgcggctgttcgatcgccccatgcaggcgccctttcttattagctcaatgaccggcggcgcggcgcgcgccctctcgattaatcatcatcttgccgaagcggcgcagacgctgggtctggcgctgggggtcggttcgcagcgcgtggcgctggaaagcgacaacgattctggcctgacgcgcgatttacgccgtatcgccccggatattccgctgctggcgaacctcggcgcggcgcagattctgggcgaacagggccgcaggctggcgcgaaatgcggtaagcatgatcgaggcggatgcgctgatcgtccatcttaatccgctgcaggaagcgctgcagcgcggcggcgatcgcgactggcgcggcgtactgcaggcgattgcgcagctggtgaagtcgctggaggtgccggtggtggtgaaagaggttggcgcgggcatctcggccgaggttgcgcagcggctcgccgaggcgggcgtcagcatgatcgatatcgcaggtgcgggcggcaccagctgggcggcggtagagggcgaacgcgccagcaccccgcagcagcgcgcggtggcgatggcctttgccagctggggtattcccacagatgaagccttacgcgcggtgcgcgacaggctgcctgccataccgcttatcgcctcaggcggcatccgcgacggcatcgacgcggcgaaggcgctgcggctcggcgcggatatcgttggccaggcggcggcggtgctcagcagcgccctgcactctacggatgcggtggtcgcgcactttaacacgctgattgaacagctgcgcgtcgcctgtttctgcaccggcagcgctaatctgcgccagctgcgccttgcgccgctgcatcgcgccggagaaacgctatga

SEQ ID NO: 11 - DNA, 1860 bp, Methylomonas spl 16a
atgaaactgaccaccgactatcccttgcttaaaaacatccacacgccggcggacatacgcgcgctgtccaaggaccagctccagcaactggctgacgaggtgcgcggctatctgacccacacggtcagcatttccggcggccattttgcggccggcctcggcaccgtggaactgaccgtggccttgcattatgtgttcaatacccccgtcgatcagttggtctgggacgtgggccatcaggcctatccgcacaagattctgaccggtcgcaaggagcgcatgccgaccattcgcaccctgggcggggtgtcagcctttccggcgcgggacgagagcgaatacgatgccttcggcgtcggccattccagcacctcgatcagcgcggcactgggcatggccattgcgtcgcagctgcgcggcgaagacaagaagatggtagccatcatcggcgacggttccatcaccggcggcatggcctatgaggcgatgaatcatgccggcgatgtgaatgccaacctgctggtgatcttgaacgacaacgatatgtcgatctcgccgccggtcggggcgatgaacaattatctgaccaaggtgttgtcgagcaagttttattcgtcggtgcgggaagagagcaagaaagctctggccaagatgccgtcggtgtgggaactggcgcgcaagaccgaggaacacgtgaagggcatgatcgtgcccggtaccttgttcgaggaattgggcttcaattatttcggcccgatcgacggccatgatgtcgagatgctggtgtcgaccctggaaaatctgaaggatttgaccgggccggtattcctgcatgtggtgaccaagaagggcaaaggctatgcgccagccgagaaagacccgttggcctaccatggcgtgccggctttcgatccgaccaaggatttcctgcccaaggcggcgccgtcgccgcatccgacctataccgaggtgttcggccgctggctgtgcgacatggcggctcaagacgagcgcttgctgggcatcacgccggcgatgcgcgaaggctctggtttggtggaattctcacagaaatttccgaatcgctatttcgatgtcgccatcgccgagcagcatgcggtgaccttggccgccggccaggcctgccagggcgccaagccggtggtggcgatttattccaccttcctgcaacgcggttacgatcagttgatccacgacgtggccttgcagaacttagatatgctctttgcactggatcgtgccggcttggtcggcccggatggaccgacccatgctggcgcctttgattacagctacatgcgctgtattccgaacatgctgatcatggctccagccgacgagaacgagtgcaggcagatgctgaccaccggcttccaacaccatggcccggcttcggtgcgctatccgcgcggcaaagggcccggggcggcaatcgatccgaccctgaccgcgctggagatcggcaaggccgaagtcagacaccacggcagccgcatcgccattctggcctggggcagcatggtcacgcctgccgtcgaagccggcaagcagctgggcgcgacggtggtgaacatgcgtttcgtcaagccgttcgatcaagccttggtgctggaattggccaggacgcacgatgtgttcgtcaccgtcgaggaaaacgtcatcgccggcggcgctggcagtgcgatcaacaccttcctgcaggcgcagaaggtgctgatgccggtctgcaacatcggcctgcccgaccgcttcgtcgagcaaggtagtcgcgaggaattgctcagcctggtcggcctcgacagcaagggcatcctcgccaccatcgaacagttttgcgct

SEQ ID NO: 12 - protein, 620 aa, Methylomonas sp. 16a
MKLTTDYPLLKNIHTPADIRALSKDQLQQLADEVRGYLTHTVSISGGHFAAGLGTVELTVALHYVFNTPVDQLVWDVGHQAYPHKILTGRKERMPTIRTLGGVSAFPARDESEYDAFGVGHSSTSISAALGMAIASQLRGEDKKMVAIIGDGSITGGMAYEAMNHAGDVNANLLVILNDNDMSISPPVGAMNNYLTKVLSSKFYSSVREESKKALAKMPSVWELARKTEEHVKGMIVPGTLFEELGFNYFGPIDGHDVEMLVSTLENLKDLTGPVFLHVVTKKGKGYAPAEKDPLAYHGVPAFDPTKDFLPKAAPSPHPTYTEVFGRWLCDMAAQDERLLGITPAMREGSGLVEFSQKFPNRYFDVAIAEQHAVTLAAGQACQGAKPVVAIYSTFLQRGYDQLIHDVALQNLDMLFALDRAGLVGPDGPTHAGAFDYSYMRCIPNMLIMAPADENECRQMLTTGFQHHGPASVRYPRGKGPGAAIDPTLTALEIGKAEVRHHGSRIAILAWGSMVTPAVEAGKQLGATVVNMRFVKPFDQALVLELARTHDVFVTVEENVIAGGAGSAINTFLQAQKVLMPVCNIGLPDRFVEQGSREELLSLVGLDSKGILATIEQFCA

SEQ ID NO: 13 - DNA, 1182 bp, Methylomonas sp. 16a
atgaaaggtatttgcatattgggcgctaccggttcgatcggtgtcagcacgctggatgtcgttgccaggcatccggataaatatcaagtcgttgcgctgaccgccaacggcaatatcgacgcattgtatgaacaatgcctggcccaccatccggagtatgcggtggtggtcatggaaagcaaggtagcagagttcaaacagcgcattgccgcttcgccggtagcggatatcaaggtcttgtcgggtagcgaggccttgcaacaggtggccacgctggaaaacgtcgatacggtgatggcggctatcgtcggcgcggccggattgttgccgaccttggccgcggccaaggccggcaaaaccgtgctgttggccaacaaggaagccttggtgatgtcgggacaaatcttcatgcaggccgtcagcgattccggcgctgtgttgctgccgatagacagcgagcacaacgccatctttcagtgcatgccggcgggttatacgccaggccatacagccaaacaggcgcgccgcattttattgaccgcttccggtggcccatttcgacggacgccgatagaaacgttgtccagcgtcacgccggatcaggccgttgcccatcctaaatgggacatggggcgcaagatttcggtcgattccgccaccatgatgaacaaaggtctcgaactgatcgaagcctgcttgttgttcaacatggagcccgaccagattgaagtcgtcattcatccgcagagcatcattcattcgatggtggactatgtcgatggttcggttttggcgcagatgggtaatcccgacatgcgcacgccgatagcgcacgcgatggcctggccggaacgctttgactctggtgtggcgccgctggatattttcgaagtagggcacatggatttcgaaaaacccgacttgaaacggtttccttgtctgagattggcttatgaagccatcaagtctggtggaattatgccaacggtattgaacgcagccaatgaaattgctgtcgaagcgtttttaaatgaagaagtcaaattcactgacatcgcggtcatcatcgagcgcagcatggcccagtttaaaccggacgatgccggcagcctcgaattggttttgcaggccgatcaagatgcgcgcgaggtggctagagacatcatcaagaccttggtagct

SEQ ID NO: 14 - protein, 394 aa, Methylomonas sp. 16a
MKGICILGATGSIGVSTLDVVARHPDKYQVVALTANGNIDALYEQCLAHHPEYAVVVMESKVAEFKQRIAASPVADIKVLSGSEALQQVATLENVDTVMAAIVGAAGLLPTLAAAKAGKTVLLANKEALVMSGQIFMQAVSDSGAVLLPIDSEHNAIFQCMPAGYTPGHTAKQARRILLTASGGPFRRTPIETLSSVTPDQAVAHPKWDMGRKISVDSATMMNKGLELIEACLLFNMEPDQIEVVIHPQSIIHSMVDYVDGSVLAQMGNPDMRTPIAHAMAWPERFDSGVAPLDIFEVGHMDFEKPDLKRFPCLRLAYEAIKSGGIMPTVLNAANEIAVEAFLNEEVKFTDIAVIIERSMAQFKPDDAGSLELVLQADQDAREVARDIIKTLVA

SEQ ID NO: 15 - DNA, 693 bp, Methylomonas sp. 16a
atgaacccaaccatccaatgctgggccgtcgtgcccgcagccggcgtcggcaaacgcatgcaagccgatcgccccaaacaatatttaccgcttgccggtaaaacggtcatcgaacacacactgactcgactacttgagtccgacgccttccaaaaagttgcggtggcgatttccgtcgaagacccttattggcctgaactgtccatagccaaacaccccgacatcatcaccgcgcctggcggcaaggaacgcgccgactcggtgctgtctgcactgaaggctttagaagatatagccagcgaaaatgattgggtgctggtacacgacgccgcccgcccctgcttgacgggcagcgacatccaccttcaaatcgataccttaaaaaatgacccggtcggcggcatcctggccttgagttcgcacgacacattgaaacacgtggatggtgacacgatcaccgcaaccatagacagaaagcacgtctggcgcgccttgacgccgcaaatgttcaaatacggcatgttgcgcgacgcgttgcaacgaaccgaaggcaatccggccgtcaccgacgaagccagtgcgctggaacttttgggccataaacccaaaatcgtggaaggccgcccggacaacatcaaaatcacccgcccggaagatttggccctggcacaattttatatggagcaacaagca

SEQ ID NO: 16 - protein, 231 aa, Methylomonas sp.16a
MNPTIQCWAVVPAAGVGKRMQADRPKQYLPLAGKTVIEHTLTRLLESDAFQKVAVAISVEDPYWPELSIAKHPDIITAPGGKERADSVLSALKALEDIASENDWVLVHDAARPCLTGSDIHLQIDTLKNDPVGGILALSSHDTLKHVDGDTITATIDRKHVWRALTPQMFKYGMLRDALQRTEGNPAVTDEASALELLGHKPKIVEGRPDNIKITRPEDLALAQFYMEQQA

SEQ ID NO: 17 - DNA, 855 bp, Methylomonas sp. 16a
atggattatgcggctgggtggggcgaaagatggcctgctccggcaaaattgaacttaatgttgaggattaccggtcgcaggccagatggctatcatctgttgcaaacggtgtttcaaatgctcgatctatgcgattggttgacgtttcatccggttgatgatggccgcgtgacgctgcgaaatccaatctccggcgttccagagcaggatgacttgactgttcgggcggctaatttgttgaagtctcataccggctgtgtgcgcggagtttgtatcgatatcgagaaaaatctgcctatgggtggtggtttgggtggtggaagttccgatgctgctacaaccttggtagttctaaatcggctttggggcttgggcttgtcgaagcgtgagttgatggatttgggcttgaggcttggtgccgatgtgcctgtgtttgtgtttggttgttcggcctggggcgaaggtgtgagcgaggatttgcaggcaataacgttgccggaacaatggtttgtcatcattaaaccggattgccatgtgaatactggagaaattttttctgcagaaaatttgacaaggaatagtgcagtcgttacaatgagcgactttcttgcaggggataatcggaatgattgttcggaagtggtttgcaagttatatcgaccggtgaaagatgcaatcgatgcgttgttatgctatgcggaagcgagattgacggggaccggtgcatgtgtgttcgctcagttttgtaacaaggaagatgctgagagtgcgttagaaggattgaaagatcggtggctggtgttcttggctaaaggcttgaatcagtctgcgctctacaagaaattagaacaggga

SEQ ID NO: 18 - protein, 285 aa, Methylomonas sp. 16a
MDYAAGWGERWPAPAKLNLMLRITGRRPDGYHLLQTVFQMLDLCDWLTFHPVDDGRVTLRNPISGVPEQDDLTVRAANLLKSHTGCVRGVCIDIEKNLPMGGGLGGGSSDAATTLVVLNRLWGLGLSKRELMDLGLRLGADVPVFVFGCSAWGEGVSEDLQAITLPEQWFVIIKPDCHVNTGEIFSAENLTRNSAVVTMSDFLAGDNRNDCSEVVCKLYRPVKDAIDALLCYAEARLTGTGACVFAQFCNKEDAESALEGLKDRWLVFLAKGLNQSALYKKLEQG

SEQ ID NO: 19 - DNA, 471 bp, Methylomonas sp. 16a
atgatacgcgtaggcatgggttacgacgtgcaccgtttcaacgacggcgaccacatcattttgggcggcgtcaaaatcccttatgaaaaaggcctggaagcccattccgacggcgacgtggtgctgcacgcattggccgacgccatcttgggagccgccgctttgggcgacatcggcaaacatttcccggacaccgaccccaatttcaagggcgccgacagcagggtgctactgcgccacgtgtacggcatcgtcaaggaaaaaggctataaactggtcaacgccgacgtgaccatcatcgctcaggcgccgaagatgctgccacacgtgcccggcatgcgcgccaacattgccgccgatctggaaaccgatgtcgatttcattaatgtaaaagccacgacgaccgagaaactgggctttgagggccgtaaggaaggcatcgccgtgcaggctgtggtgttgatagaacgc

SEQ ID NO: 20 - protein, 157 aa, Methylomonas sp. 16a
MIRVGMGYDVHRFNDGDHIILGGVKIPYEKGLEAHSDGDVVLHALADAILGAAALGDIGKHFPDTDPNFKGADSRVLLRHVYGIVKEKGYKLVNADVTIIAQAPKMLPHVPGMRANIAADLETDVDFINVKATTTEKLGFEGRKEGIAVQAVVLIER

SEQ ID NO: 21 - DNA, 1218 bp, Methylomonas sp. 16a
atgtcgattagccccagaaaacacacacaacaggttttggttggtaacattaaagtcggtggcggcgccccgattgtcgtgcaatcgatgaccaataccgataccgccgatgttgcaggaagcgttcaacagatcatggaattagccacggctggctctgaagtagtcagagtaaccgtcaacaccgaggaagcagccaaagccgtgcccgaaatcgtcaaccagctggcgcaaaaaggcttcgacacgcccattatcggcgacttccatttcaacggccacaagttactggaaaaataccccgactgcgcgcaagccctggccaaataccgcatcaatcccggcaacgtcggcaaaggcaaggcccgcgatccgcaattccagcaaatgatcgaattcgcgatccaatacaacaaacccgtgcgcatcggtgtcaacgggggcagcttggatcaggcggtgctgacacgcttgctggatgaaaaccgtcaattggccgaacccaaggaattgcccgccattacccgcgaagccatcgtgctgtctgccctggaaagcgccgccaaagctcaggaaatcggcctgggtaaagacaaaatcattctgtcctgcaaaatcagcaacgtacaggaactgatcagcatctacgaagacctttccaaccgctgcgactatgccttgcacctgggcctgaccgaagccggcatgggctccaaaggcatcgtagcttccgccgccgccttgggcgtattgatgcaacaaggcataggcgacaccatccgcatctcattgacccccgaacccggcgccgcacgcaccaaggaagtcatcgtagcgcaagaaatcctgcaaaccatgggcttccgctcgttcacgccgatggttatcgcctgccccggctgcggccgcaccaccagcgattacttccaacgcctggcacaagacatccaagcatttttacgcgaacaaatgccggtctggaaaaccaaatacaaaggcgtggaaaacatggaagtcgcagtcatggggtgcgtcgtgaatggccccggcgaaagcaagaacgccaacatcggcatcagcctgcccggcagcggcgaatccccggtcgcgccggtttatgaagacggtgtcaaaaccgtcaccttgaaaggcgacaacattgcagccgagttccagactttagtagaaaaatacatcgaatcgcattacggcgcccat

SEQ ID NO: 22 - protein, 406 aa, Methylomonas sp. 16a
MSISPRKHTQQVLVGNIKVGGGAPIVVQSMTNTDTADVAGSVQQIMELATAGSEVVRVTVNTEEAAKAVPEIVNQLAQKGFDTPIIGDFHFNGHKLLEKYPDCAQALAKYRINPGNVGKGKARDPQFQQMIEFAIQYNKPVRIGVNGGSLDQAVLTRLLDENRQLAEPKELPAITREAIVLSALESAAKAQEIGLGKDKIILSCKISNVQELISIYEDLSNRCDYALHLGLTEAGMGSKGIVASAAALGVLMQQGIGDTIRISLTPEPGAARTKEVIVAQEILQTMGFRSFTPMVIACPGCGRTTSDYFQRLAQDIQAFLREQMPVWKTKYKGVENMEVAVMGCVVNGPGESKNANIGISLPGSGESPVAPVYEDGVKTVTLKGDNIAAEFQTLVEKYIESHYGAH

SEQ ID NO: 23 - DNA, 954 bp, Methylomonas sp. 16a
atgcaaatcgtactcgcaaacccccgtggattctgtgccggcgtggaccgggccattgaaattgtcgatcaagccatcgaagcctttggtgcgccgatttatgtgcggcacgaggtggtgcataaccgcaccgtggtcgatggactgaaacaaaaaggtgcggtgttcatcgaggaactaagcgatgtgccggtgggttcctacttgattttcagcgcgcacggcgtatccaaggaggtgcaacaggaagccgaggagcgccagttgacggtattcgatgcgacttgtccgctggtgaccaaagtgcacatgcaggttgccaagcatgccaaacagggccgagaagtgattttgatcggccacgccggtcatccggaagtggaaggcacgatgggccagtatgaaaaatgcaccgaaggcggcggcatttatctggtcgaaactccggaagacgtacgcaatttgaaagtcaacaatcccaatgatctggcctatgtgacgcagacgaccttgtcgatgaccgacaccaaggtcatggtggatgcgttacgcgaacaatttccgtccattaaggagcaaaaaaaggacgatatttgttacgcgacgcaaaaccgtcaggatgcggtgcatgatctggccaagatttccgacctgattctggttgtcggctctcccaatagttcgaattccaaccgtttgcgtgaaatcgccgtgcaactcggtaaacccgcttatttgatcgatacttaccaggatttgaagcaagattggctggagggaattgaagtagtcggggttaccgcgggcgcttcggcgccggaagtgttggtgcaggaagtgatcgatcaactgaaggcatggggcggcgaaaccacttcggtcagagaaaacagcggcatcgaggaaaaggtagtcttttcgattcccaaggagttgaaaaaacatatgcaagcg

SEQ ID NO: 24 - protein, 318 aa, Methylomonas sp. 16a
MQIVLANPRGFCAGVDRAIEIVDQAIEAFGAPIYVRHEVVHNRTVVDGLKQKGAVFIEELSDVPVGSYLIFSAHGVSKEVQQEAEERQLTVFDATCPLVTKVHMQVAKHAKQGREVILIGHAGHPEVEGTMGQYEKCTEGGGIYLVETPEDVRNLKVNNPNDLAYVTQTTLSMTDTKVMVDALREQFPSIKEQKKDDICYATQNRQDAVHDLAKISDLILVVGSPNSSNSNRLREIAVQLGKPAYLIDTYQDLKQDWLEGIEVVGVTAGASAPEVLVQEVIDQLKAWGGETTSVRENSGIEEKVVFSIPKELKKHMQA

SEQ ID NO: 25 - DNA, 891 bp, Methylomonas sp. 16a
atgagtaaattgaaagcctacctgaccgtctgccaagaacgcgtcgagcgcgcgctggacgcccgtctgcctgccgaaaacatactgccacaaaccttgcatcaggccatgcgctattccgtattgaacggcggcaaacgcacccggcccttgttgacttatgcgaccggtcaggctttgggcttgccggaaaacgtgctggatgcgccggcttgcgcggtagaattcatccatgtgtattcgctgattcacgacgatctgccggccatggacaacgatgatctgcgccgcggcaaaccgacctgtcacaaggcttacgacgaggccaccgccattttggccggcgacgcactgcaggcgctggcctttgaagttctggccaacgaccccggcatcaccgtcgatgccccggctcgcctgaaaatgatcacggctttgacccgcgccagcggctctcaaggcatggtgggcggtcaagccatcgatctcggctccgtcggccgcaaattgacgctgccggaactcgaaaacatgcatatccacaagactggcgccctgatccgcgccagcgtcaatctggcggcattatccaaacccgatctggatacttgcgtcgccaagaaactggatcactatgccaaatgcataggcttgtcgttccaggtcaaagacgacattctcgacatcgaagccgacaccgcgacactcggcaagactcagggcaaggacatcgataacgacaaaccgacctaccctgcgctattgggcatggctggcgccaaacaaaaagcccaggaattgcacgaacaagcagtcgaaagcttaacgggatttggcagcgaagccgacctgctgcgcgaactatcgctttacatcatcgagcgcacgcac

SEQ ID NO: 26 - protein, 297 aa, Methylomonas sp. 16a
MSKLKAYLTVCQERVERALDARLPAENILPQTLHQAMRYSVLNGGKRTRPLLTYATGQALGLPENVLDAPACAVEFIHVYSLIHDDLPAMDNDDLRRGKPTCHKAYDEATAILAGDALQALAFEVLANDPGITVDAPARLKMITALTRASGSQGMVGGQAIDLGSVGRKLTLPELENMHIHKTGALIRASVNLAALSKPDLDTCVAKKLDHYAKCIGLSFQVKDDILDIEADTATLGKTQGKDIDNDKPTYPALLGMAGAKQKAQELHEQAVESLTGFGSEADLLRELSLYIIERTH

SEQ ID NO: 27 - DNA, 1533 bp, Methylomonas sp. 16a
atggccaacaccaaacacatcatcatcgtcggcgcgggtcccggcggactttgcgccggcatgttgctgagccagcgcggcttcaaggtatcgattttcgacaaacatgcagaaatcggcggccgcaaccgcccgatcaacatgaacggctttaccttcgataccggtccgacattcttgttgatgaaaggcgtgctggacgaaatgttcgaactgtgcgagcgccgtagcgaggattatctggaattcctgccgctaagcccgatgtaccgcctgctgtacgacgaccgcgacatcttcgtctattccgaccgcgagaacatgcgcgccgaattgcaacgggtattcgacgaaggcacggacggctacgaacagttcatggaacaggaacgcaaacgcttcaacgcgctgtatccctgcatcacccgcgattattccagcctgaaatcctttttgtcgctggacttgatcaaggccctgccgtggctggcttttccgaaaagcgtgttcaataatctcggccagtatttcaaccaggaaaaaatgcgcctggccttttgctttcagtccaagtatctgggcatgtcgccgtgggaatgcccggcactgtttacgatgctgccctatctggagcacgaatacggcatttatcacgtcaaaggcggcctgaaccgcatcgcggcggcgatggcgcaagtgatcgcggaaaacggcggcgaaattcacttgaacagcgaaatcgagtcgctgatcatcgaaaacggcgctgccaagggcgtcaaattacaacatggcgcggagctgcgcggcgacgaagtcatcatcaacgcggattttgcccacgcgatgacgcatctggtcaaaccgggcgtcttgaaaaaatacaccccggaaaacctgaagcagcgcgagtattcctgttcgaccttcatgctgtatctgggtttggacaagatttacgatctgccgcaccataccatcgtgtttgccaaggattacaccaccaatatccgcaacattttcgacaacaaaaccctgacggacgatttttcgttttacgtgcaaaacgccagcgccagcgacgacagcctagcgccagccggcaaatcggcgctgtacgtgctggtgccgatgcccaacaacgacagcggcctggactggcaggcgcattgccaaaacgtgcgcgaacaggtgttggacacgctgggcgcgcgactgggattgagcgacatcagagcccatatcgaatgcgaaaaaatcatcacgccgcaaacctgggaaacggacgaacacgtttacaagggcgccactttcagtttgtcgcacaagttcagccaaatgctgtactggcggccgcacaaccgtttcgaggaactggccaattgctatctggtcggcggcggcacgcatcccggtagcggtttgccgaccatctacgaatcggcgcggatttcggccaagctgatttcccagaaacatcgggtgaggttcaaggacatagcacacagcgcctggctgaaaaaagccaaagcc

SEQ ID NO: 28 - protein, 511 aa, Methylomonas sp. 16a
MANTKHIIIVGAGPGGLCAGMLLSQRGFKVSIFDKHAEIGGRNRPINMNGFTFDTGPTFLLMKGVLDEMFELCERRSEDYLEFLPLSPMYRLLYDDRDIFVYSDRENMRAELQRVFDEGTDGYEQFMEQERKRFNALYPCITRDYSSLKSFLSLDLIKALPWLAFPKSVFNNLGQYFNQEKMRLAFCFQSKYLGMSPWECPALFTMLPYLEHEYGIYHVKGGLNRIAAAMAQVIAENGGEIHLNSEIESLIIENGAAKGVKLQHGAELRGDEVIINADFAHAMTHLVKPGVLKKYTPENLKQREYSCSTFMLYLGLDKIYDLPHHTIVFAKDYTTNIRNIFDNKTLTDDFSFYVQNASASDDSLAPAGKSALYVLVPMPNNDSGLDWQAHCQNVREQVLDTLGARLGLSDIRAHIECEKIITPQTWETDEHVYKGATFSLSHKFSQMLYWRPHNRFEELANCYLVGGGTHPGSGLPTIYESARISAKLISQKHRVRFKDIAHSAWLKKAKA

SEQ ID NO: 29 - DNA, 1491 bp, Methylomonas sp. 16a
atgaactcaaatgacaaccaacgcgtgatcgtgatcggcgccggcctcggcggcctgtccgccgctatttcgctggccacggccggcttttccgtgcaactcatcgaaaaaaacgacaaggtcggcggcaagctcaacatcatgaccaaagacggctttaccttcgatctggggccgtccattttgacgatgccgcacatctttgaggccttgttcacaggggccggcaaaaacatggccgattacgtgcaaatccagaaagtcgaaccgcactggcgcaatttcttcgaggacggtagcgtgatcgacttgtgcgaagacgccgaaacccagcgccgcgagctggataaacttggccccggcacttacgcgcaattccagcgctttctggactattcgaaaaacctctgcacggaaaccgaagccggttacttcgccaagggcctggacggcttttgggatttactcaagttttacggcccgctccgcagcctgctgagtttcgacgtcttccgcagcatggaccagggcgtgcgccgctttatttccgatcccaagttggtcgaaatcctgaattacttcatcaaatacgtcggctcctcgccttacgatgcgcccgccttgatgaacctgctgccttacattcaatatcattacggcctgtggtacgtgaaaggcggcatgtatggcatggcgcaggccatggaaaaactggccgtggaattgggcgtcgagattcgtttagatgccgaggtgtcggaaatccaaaaacaggacggcagagcctgcgccgtaaagttggcgaacggcgacgtgctgccggccgacatcgtggtgtcgaacatggaagtgattccggcgatggaaaaactgctgcgcagcccggccagcgaactgaaaaaaatgcagcgcttcgagcctagctgttccggcctggtgctgcacttgggcgtggacaggctgtatccgcaactggcgcaccacaatttcttttattccgatcatccgcgcgaacatttcgatgcggtattcaaaagccatcgcctgtcggacgatccgaccatttatctggtcgcgccgtgcaagaccgaccccgcccaggcgccggccggctgcgagatcatcaaaatcctgccccatatcccgcacctcgaccccgacaaactgctgaccgccgaggattattcagccttgcgcgagcgggtgctggtcaaactcgaacgcatgggcctgacggatttacgccaacacatcgtgaccgaagaatactggacgccgctggatattcaggccaaatattattcaaaccagggctcgatttacggcgtggtcgccgaccgcttcaaaaacctgggtttcaaggcacctcaacgcagcagcgaattatccaatctgtatttcgtcggcggcagcgtcaatcccggcggcggcatgccgatggtgacgctgtccgggcaattggtgagggacaagattgtggcggatttgcaa

SEQ ID NO: 30 - protein, 497 aa, Methylomonas sp. 16a
MNSNDNQRVIVIGAGLGGLSAAISLATAGFSVQLIEKNDKVGGKLNIMTKDGFTFDLGPSILTMPHIFEALFTGAGKNMADYVQIQKVEPHWRNFFEDGSVIDLCEDAETQRRELDKLGPGTYAQFQRFLDYSKNLCTETEAGYFAKGLDGFWDLLKFYGPLRSLLSFDVFRSMDQGVRRFISDPKLVEILNYFIKYVGSSPYDAPALMNLLPYIQYHYGLWYVKGGMYGMAQAMEKLAVELGVEIRLDAEVSEIQKQDGRACAVKLANGDVLPADIVVSNMEVIPAMEKLLRSPASELKKMQRFEPSCSGLVLHLGVDRLYPQLAHHNFFYSDHPREHFDAVFKSHRLSDDPTIYLVAPCKTDPAQAPAGCEIIKILPHIPHLDPDKLLTAEDYSALRERVLVKLERMGLTDLRQHIVTEEYWTPLDIQAKYYSNQGSIYGVVADRFKNLGFKAPQRSSELSNLYFVGGSVNPGGGMPMVTLSGQLVRDKIVADLQ

SEQ ID NO: 31 - DNA, 1542 bp, Methylomonas sp. 16a
atgcgcggcattcatccagaatccaaacacgaacaatacgacgtgatcgtcataggcgccggtatcggcggcttgagcacggccgcgttattggcgaaagccggaaaagccgtgttgttagtcgaaaggcatgaccgccccggcggttatgctcacggtttccggcggcgcaattaccatttcgattcgggggtacatctggtcagcggctgcggtgccgacggctatgaaaacggcagcacgatttaccggatttgccgggccgtgggcatagaccccgaggatgttttccttccgatcccgtcttacgcccgcgcggtgtttccggggttcgaactgagcctgcatgccggcgaagaggtgttcgtcggtgagttatgcgcgcatttcccaaacgaaaaggacaatctgctccgcttgattcggctctgcaaaaccctggcggaagaagccatgctggcggaagaaattctggaacagagcaaaatcactcgcgtaccacccacgcgagcgctggccaatttgtttcgttaccgccgcgccaccttggcggaagcactggatgaatttttgctcgacccacatcttaaaagtgcctgcgccgcgctatggccttatttgggcctaccgccttcgcaactgtctttcttatattgggccagcatgatggcgggctacacctacgaaggtgcgtattattgccgcggcagttttcaaacctatgccaacagactggcgcaagcgatcgaaaagcgcggcggcgaggtgttattgaacgccagcgtgcggcggatttgcgtggaaaacggcggcatcagcggcatcatgctggaaaatggtcaactaatacgcgcaaagaccgtagtctcgaatgtcgccgcccagcaaaccgccgaattactgatcggtcgcgagcattggccggctggctattgcgacaagctggaaaagttggcgccgtcgctgtcgattttcgccagctacatcgcaaccgatttggccatcgacacggccgttcatagccacgagtcgtttttttaccaaaccttcgatcacgaagccgggtttgcatccacgcacaaggggcagcccaattggttttcggccaccctgtcgacgttgagcgatgcctcgctggcaccggccggtcaacacaccctgatgctgaccaccttatgcccgtttgacatagggcaaagctggcgacaggccaaactggactttgagcaacgcttattggcgcaagccgaacaacattttccaggcctgaaagaccatttgttgctgatagaatccggctcgccgcgcacgctggaacgctacaccctcaaccaccaaggcgcggcctacggctttgcccctacccccgatcaaatcggcccaaaccgtccggacgttcgcggagccttgccgggcttgttccacaccggccactggacgcgtccgggcggcggcgtcgccggcgtcagtatctcggctcaactggcggcacaagccattttgaacctgcccatacaagccgatttctggaacagcctggat

SEQ ID NO: 32 - protein, 514 aa, Methylomonas sp. 16a
MRGIHPESKHEQYDVIVIGAGIGGLSTAALLAKAGKAVLLVERHDRPGGYAHGFRRRNYHFDSGVHLVSGCGADGYENGSTIYRICRAVGIDPEDVFLPIPSYARAVFPGFELSLHAGEEVFVGELCAHFPNEKDNLLRLIRLCKTLAEEAMLAEEILEQSKITRVPPTRALANLFRYRRATLAEALDEFLLDPHLKSACAALWPYLGLPPSQLSFLYWASMMAGYTYEGAYYCRGSFQTYANRLAQAIEKRGGEVLLNASVRRICVENGGISGIMLENGQLIRAKTVVSNVAAQQTAELLIGREHWPAGYCDKLEKLAPSLSIFASYIATDLAIDTAVHSHESFFYQTFDHEAGFASTHKGQPNWFSATLSTLSDASLAPAGQHTLMLTTLCPFDIGQSWRQAKLDFEQRLLAQAEQHFPGLKDHLLLIESGSPRTLERYTLNHQGAAYGFAPTPDQIGPNRPDVRGALPGLFHTGHWTRPGGGVAGVSISAQLAAQAILNLPIQADFWNSLD

SEQ ID NO: 33 - DNA, 627 bp, Methylomonas sp. 16a
atgtccgtcaccatcaaagaagtcatgaccacctcgcccgttatgccggtcatggtcatcaatcatctggaacatgccgtccctctggctcgcgcgctagtcgacggtggcttgaaagttttggagatcacattgcgcacgccggtggcactggaatgtatccgacgtatcaaagccgaagtaccggacgccatcgtcggcgcgggcaccatcatcaaccctcataccttgtatcaagcgattgacgccggtgcggaattcatcgtcagccccggcatcaccgaaaatctactcaacgaagcgctagcatccggcgtgcctatcctgcccggcgtcatcacacccagcgaggtcatgcgtttattggaaaaaggcatcaatgcgatgaaattctttccggctgaagccgccggcggcataccgatgctgaaatcccttggcggccccttgccgcaagtcaccttctgtccgaccggcggcgtcaatcccaaaaacgcgcccgaatatctggcattgaaaaatgtcgcctgcgtcggcggctcctggatggcgccggccgatctggtagatgccgaagactgggcggaaatcacgcggcgggcgagcgaggccgcggcattgaaaaaa

SEQ ID NO: 34 - protein, 209 aa, methylomonas sp. 16a
MSVTIKEVMTTSPVMPVMVINHLEHAVPLARALVDGGLKVLEITLRTPVALECIRRIKAEVPDAIVGAGTIINPHTLYQAIDAGAEFIVSPGITENLLNEALASGVPILPGVITPSEVMRLLEKGINAMKFFPAEAAGGIPMLKSLGGPLPQVTFCPTGGVNPKNAPEYLALKNVACVGGSWMAPADLVDAEDWAEITRRASEAAALKK

SEQ ID NO: 35 - DNA, 1431 bp, Methylomonas sp. 16a
atgaaaaaaatcctgttcgccagtagcgaaacccatcccttgatcaaaaccggcgggctggccgatgtcgccggcagcctgccgattgcgctgaccagcctagaccaggacgtgcgcgtcatcatgccccattatcaaggcatcaaaaactgcgagcccggtcgttatttgtgcacggtgcgggtcaacaattgcgacgtcaacctgctggaaacgcatttaccggaaagcgatgtcatcgtttggctggtcgattatccgccgttcttcaatcatccaggcaatccataccatgatgaaaacggcacgccctggcccgacatcggcgaccgtttcgcgctgttttgccgcatcgtggtcgaagtagcgatgaaccgggcctacttggactggaaaccggacgtcgtgcattgcaacgactggcaaaccggcctggtccctgctctgctgtcattggaagagcataggccggccacggtgtttacgatacataacatggcttaccagggtgtctttcccagcaatgcctacactttgctgaatctgcctggccagctctggcatccggacggtctggaataccatggcatgctgtccttcatcaagggcggcttgagctattccgactggatcaccaccgtcagcccgacctatgcccaggaaatccagaccccggaattcggttacggcctggaaggcttgttggcccaccggcagccgaccttgtccggcatcatcaacgggattgacaccaaagtctggaatccggaaaccgaccccttcatcgcgcaaacttacagcggcaagactctcggcaaaaaagtgctgaacaaaaccgcattgcaggcgcgtttgggattaccggtcaacgcggatttgccgctgttgggcttgatcggccgactggtcgatcagaaaggcatagacctggtgctgggctgcttgaaggaattggtcaacatgcccttgcagttcgccttgctcggcagcggcgacaacagcatacaggtgcgtttgcaggatttcgcccgcctgtatccggagaaggtttcggtgaccatcgggtacgacgaaaatctggcccaccaaatcgaagccggctccgacctgtttctgatgccgtcgcgcttcgaaccctgcggcttgaaccagatgtatagccagcgttacggtaccctgcccgtcgtcagaaaaaccggcggtctggccgacaccgtggtagacacattgcccgacacgatcaaaaacggcaccgcgagcggattcgtcttcaacgatgccgtaccgggcgcgttactggaaaccatcaagcgcgcgctggtggtgcacgccaatcccaagctctggcaagagctgcagcgcaacgcgatgcgcaaggatttttcctgggaaaacagcgccaatcagtatctggcgttatatcgtgaaatc

SEQ ID NO: 36 - protein, 477 aa, Methylomonas sp. 16a
MKKILFASSETHPLIKTGGLADVAGSLPIALTSLDQDVRVIMPHYQGIKNCEPGRYLCTVRVNNCDVNLLETHLPESDVIVWLVDYPPFFNHPGNPYHDENGTPWPDIGDRFALFCRIVVEVAMNRAYLDWKPDVVHCNDWQTGLVPALLSLEEHRPATVFTIHNMAYQGVFPSNAYTLLNLPGQLWHPDGLEYHGMLSFIKGGLSYSDWITTVSPTYAQEIQTPEFGYGLEGLLAHRQPTLSGIINGIDTKVWNPETDPFIAQTYSGKTLGKKVLNKTALQARLGLPVNADLPLLGLIGRLVDQKGIDLVLGCLKELVNMPLQFALLGSGDNSIQVRLQDFARLYPEKVSVTIGYDENLAHQIEAGSDLFLMPSRFEPCGLNQMYSQRYGTLPVVRKTGGLADTVVDTLPDTIKNGTASGFVFNDAVPGALLETIKRALVVHANPKLWQELQRNAMRKDFSWENSANQYLALYREI

SEQ ID NO: 37 - DNA, 5624 bp, artificial sequence
  constructed vector
gatcgctagtttgttttgactccatccattagggcttctaaaacgccttctaaggccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctggatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgatgaccaggtcgaattcgagctcggtaccgatcttaacatttttcccctatcatttttccgtcttcatttgtcattttttccagaaaaaatcgcgtcattcgactcatgtctaatccaacacgtgtctctcggcttatcccctgacaccgcccgccgacagcccgcatgggacgattctatcaattcagccgcggagtctagttttatattgcagaatgcgagattgctggtttattataacaatataagttttcattattttcaaaaagggggatttattgtgggtttaggtaagaaattgtctgttgctgtcgccgcttcctttatgagtttaaccatcagtctgccgggtgttcaggccgctgaggatatcaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtccgacagctggccgctgcaaaacgctgacggaacagtagcagaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccgccgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttggcccaagcttcttaatcaacatcaaaggcaataaaacatccgttgtcaaaaacagcatcctggagcaaggacagctgacagtcaactaataacagcgacatcgatgtctactggcttaactatgcggcatcagagcagattgtactgagagtgcactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgggatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcattgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctggctagcgtgatggatatctgcagaattcgcccttaaaatgccgccagcggaactggcggctgtgggattaaagggcgaattccagcacactggcggccgttactagtggatccgagctcggtaccaagcttgatgcagatctgcaggtcgacggatcccaagcttcttctagaggtaccgcatgcgatatcgagctctcccgggaattc

SEQ ID NO: 38 - DNA, 1203 bp, artificial sequence
  constructed DNS fragment
attcgtgttgccggtgccgatgatgaacatcatcaacggcggttcgcacgccgacaacagcgtcgatttgcaagaattcatgattttgcctgttggtgcgccgactttccgcgaagccatccgttacggcgccgaggtgttccacaacctggctaaagtcttgaaaggcaaaggtctggccactaccgtcggcgacgaaggcggtttcgcccctaacctgcgttccaacgaagaagccatcgaggtcatcctggaagcgatcgaaaaagccggctacaaagcaggccaagacatctatctgggtatggacgctgccgcttccgaatatttcgaaaacggcaaatacttcctgtccgccgaaaacagaagcttcaattccgaagaaatggtcgacttcctggcggcttgggtagacaaatatcccatcatttcgatcgaagacggcctggacgaaaacgattgggacggctggaaatatcaaaccgaaaaactgggtaaccgcattcaattggtcggcgacgacttgttcgtaaccaaccctgcgatcctaaaagaaggcatcgagaaaggcatcgccaactcgatcctgatcaaggtcaaccaaatcggcacgctgaccgaaaccttggccgcgatcgacatggccaaggccgcgggttacagcgccgtggtttctcaccggtccggcgaaaccgaagacaccaccatcgcggacctggtcgtcgccaccggcaccggccaaatcaaaaccggttcactgagccgttccgaccgggtcgccaaatacaaccgtctgatgaaaatcgaagacgagttgggcgccaaagcccgttacgctggccgcagcgccttcaaaatgctgggttaatattcagcttgtacgaaccgggttatgctattgggcataacccggcctttaccgagataccgctatcaaatccatcatcatcctcatcattgcgctgatcatccacttccagtatcggttgtggtttggtgacgcaagcgttgcacaaatcggcgaatatcgcgagcgcctggaggaattgaacaaggaaatacaggaaaaacaggagcgcaatgacgcgctgtacgccgaggtattggatttacgccgcggcctggaaaccatagaggaacgcgcccgttacgaactgggcatgatcaaggaaaacgaaaccttctttcaagtgctggaatagc

SEQ ID NO: 39 - DNA, 47 bp, artificial sequence
  primer
aattcatgaaactagttctagaattcgtgttgccggtgccgatgatg

SEQ ID NO: 40 - DNA, 29 bp, artificial sequence
  primer
gctattccagcacttgaaagaaggtttcg

SEQ ID NO: 41 - DNA, 58 bp, artificial sequence
  primer
ggaaaacgaaaccttctttcaagtgctggaatagccctgtgacggaagatcacttcgc

SEQ ID NO: 42 - DNA, 50 bp, artificial sequence
  primer
gacggcccagcattggatggttgggttcattttagcttccttagctcctg

SEQ ID NO: 43 - DNA, 1225 bp, artificial sequence
  constructed fragment
atgaacccaaccatccaatgctgggccgtcgtgcccgcagccggcgtcggcaaacgcatgcaagccgatcgccccaaacaatatttaccgcttgccggtaaaacggtcatcgaacacacactgactcgactacttgagtccgacgccttccaaaaagttgcggtggcgatttccgtcgaagacccttattggcctgaactgtccatagccaaacaccccgacatcatcaccgcgcctggcggcaaggaacgcgccgactcggtgctgtctgcactgaaggctttagaagatatagccagcgaaaatgattgggtgctggtacacgacgccgcccgcccctgcttgacgggcagcgacatccaccttcaaatcgataccttaaaaaatgacccggtcggcggcatcctggccttgagttcgcacgacacattgaaacacgtggatggtgacacgatcaccgcaaccatagacagaaagcacgtctggcgcgccttgacgccgcaaatgttcaaatacggcatgttgcgcgacgcgttgcaacgaaccgaaggcaatccggccgtcaccgacgaagccagtgcgctggaacttttgggccataaacccaaaatcgtggaaggccgcccggacaacatcaaaatcacccgcccggaagatttggccctggcacaattttatatggagcaacaagcatgatacgcgtaggcatgggttacgacgtgcaccgtttcaacgacggcgaccacatcattttgggcggcgtcaaaatcccttatgaaaaaggcctggaagcccattccgacggcgacgtggtgctgcacgcattggccgacgccatcttgggagccgccgctttgggcgacatcggcaaacatttcccggacaccgaccccaatttcaagggcgccgacagcagggtgctactgcgccacgtgtacggcatcgtcaaggaaaaaggctataaactggtcaacgccgacgtgaccatcatcgctcaggcgccgaagatgctgccacacgtgcccggcatgcgcgccaacattgccgccgatctggaaaccgatgtcgatttcattaatgtaaaagccacgacgaccgagaaactgggctttgagggccgtaaggaaggcatcgccgtgcaggctgtggtgttgatagaacgctagcgcttttatgcaggaaaaaactaaagctttttcagtcctgaattggcgtgcaacagctt

SEQ ID NO: 44 - DNA, 28 bp, artificial sequence
  primer
atgaaactgaccaccgactatcccttgc

SEQ ID NO: 45 - DNA, 47 bp, artificial sequence
  primer
catctaatcaggtaccagatctaagctgttgcacgccaattcaggac

SEQ ID NO: 46 - DNA, 1868 bp, artificial sequence
  constructed fragment
gttgtcgttcaactccaaggtcgaactggacttgtcggaagtgtttctggtcagcgccggcgacgtgatgacaccgatcaaagccattcccggcgaaaagcctggccaggttttactggaattgccctcgttgattccgggcgaatatgcaatcaaattgaaaatcttcgccgcggacggtcacttgagcgaagacctgctgcgtttttttgtcaaagaagctaaataaactgtatggaaggcatagccaattatcttgactcactgatcggcggcgtcgatctgacgttttactccatcaccatcggcggcctgatgtggggcctgttcgtactgcgtccgtgggatgaaaacgccaattacaacaacgccctgctggaaaaaaccatcggcctgatttatttcggcagcaaagccttggtgatcacccagctatccaaaatcggcctgaaaatctggttgatggcggtcaccctgggtaaatcgccctttccggcatttttccagaccgtgcagtttcaagccggattggcgcgcgccgccttcgcgtttggtttattcctgttcatcaagcacagcctttacaacaacacacgctccaagcaacattggctgacggcgatcgccatcatcgtcccgctggtcatggcggctgcctggttggtacacggcgccagccgcctggaagaccgcggcttattgatgacgctgaccgtcagccaccagctcgccgccgcaacctgggtaggcggcatctttcagatcctggcgatctggcgcctgaaaaagcgcaacgccatcagtgtcgagctatggcctttgttgctgaaacgcttctccatcgtcggtatctgggcggttgtcggcctgctgatcaccggcacgccgctggcctggtattacatccgcaccttccagggcttcatcggcgccggttacggcaatctactgatggtaaagatcatgatgatgggcttggcgctcggctttgcctggatgaatcgccaagcggtcgaagactatttcagcagccgcagcctttacgtattgaccacacgcgtaccctattacatcgaagccgaaaccctgatcctgctgacgattctgttcaccgccgccagcctggcttcgcagccgccttccgtcgatattccgcatctgaccgcaacctgggaagaagtgttgaacatgttccatccgcgcatcccgcgctgggaatcgccaactcatgaagcgctgattgcaggcgaagcgggccgggtcgcgatcgtcggtcaagtcccttccgaagccgccacggcctggtcggattataaccataacatttccggcattttcctgaccgtgatgagcttcttcgcgatgctgtcctaccgggatcgctttcatcactgggcgcgctactggccagtgggtttcatgggtctgggcgtatttttgttcttccgcagcgacgcggaaagctggccgcttggccctatcggcttctgggacagcacctttaacaatggtgagatcctgcagcaccgtatcgccacactgctggtattcgtactcggctggtttgaaacccgtgcgcgcgtcaaggatgatcccggcatcttgcccttcttctttccgttactggcggccttcggcggcatgatgttgctggcacactcccatgtcggcttcgaagccaaaaccgcgttcttgatccaggtcggccataccttgatgggcgtattctcgctgatcctggcctgcggtcgctggctggaactcaagctcgattctcccggcaaaaatattgccggttttatttcagtgttcgccttgtttcaaatcggcgtcatcctgatgttctaccgtgaacccttgtactgatt

SEQ ID NO: 47 - DNA, 47 bp, artificial sequence
  primer
tcaaccaggcgctagctctagagttgtcgttcaactccaaggtcgaa

SEQ ID NO: 48 - DNA, 33 bp, artificial sequence
  primer
aatcagtacaagggttcacggtagaacatcagg

SEQ ID NO: 49 - DNA, 264 bp, Methylomonas sp. 16a
taaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatcac

SEQ ID NO: 50 - DNA, 59 bp, artificial sequence
  primer
cctgatgttctaccgtgaacccttgtactgatttaaggattggggtgcgtcgccggtcg

SEQ ID NO: 51 - DNA, 55 bp, artificial sequence
  primer
aagcaagggatagtcggtggtcagtttcattgccatgtgatgtgctccgaaagtt

SEQ ID NO: 52 - DNA, 1894 bp, artificial sequence
  constructed fragment
atgaaactgaccaccgactatcccttgcttaaaaacatccacacgccggcggacatacgcgcgctgtccaaggaccagctccagcaactggctgacgaggtgcgcggctatctgacccacacggtcagcatttccggcggccattttgcggccggcctcggcaccgtggaactgaccgtggccttgcattatgtgttcaatacccccgtcgatcagttggtctgggacgtgggccatcaggcctatccgcacaagattctgaccggtcgcaaggagcgcatgccgaccattcgcaccctgggcggggtgtcagcctttccggcgcgggacgagagcgaatacgatgccttcggcgtcggccattccagcacctcgatcagcgcggcactgggcatggccattgcgtcgcagctgcgcggcgaagacaagaagatggtagccatcatcggcgacggttccatcaccggcggcatggcctatgaggcgatgaatcatgccggcgatgtgaatgccaacctgctggtgatcttgaacgacaacgatatgtcgatctcgccgccggtcggggcgatgaacaattatctgaccaaggtgttgtcgagcaagttttattcgtcggtgcgggaagagagcaagaaagctctggccaagatgccgtcggtgtgggaactggcgcgcaagaccgaggaacacgtgaagggcatgatcgtgcccggtaccttgttcgaggaattgggcttcaattatttcggcccgatcgacggccatgatgtcgagatgctggtgtcgaccctggaaaatctgaaggatttgaccgggccggtattcctgcatgtggtgaccaagaagggcaaaggctatgcgccagccgagaaagacccgttggcctaccatggcgtgccggctttcgatccgaccaaggatttcctgcccaaggcggcgccgtcgccgcatccgacctataccgaggtgttcggccgctggctgtgcgacatggcggctcaagacgagcgcttgctgggcatcacgccggcgatgcgcgaaggctctggtttggtggaattctcacagaaatttccgaatcgctatttcgatgtcgccatcgccgagcagcatgcggtgaccttggccgccggccaggcctgccagggcgccaagccggtggtggcgatttattccaccttcctgcaacgcggttacgatcagttgatccacgacgtggccttgcagaacttagatatgctctttgcactggatcgtgccggcttggtcggcccggatggaccgacccatgctggcgcctttgattacagctacatgcgctgtattccgaacatgctgatcatggctccagccgacgagaacgagtgcaggcagatgctgaccaccggcttccaacaccatggcccggcttcggtgcgctatccgcgcggcaaagggcccggggcggcaatcgatccgaccctgaccgcgctggagatcggcaaggccgaagtcagacaccacggcagccgcatcgccattctggcctggggcagcatggtcacgcctgccgtcgaagccggcaagcagctgggcgcgacggtggtgaacatgcgtttcgtcaagccgttcgatcaagccttggtgctggaattggccaggacgcacgatgtgttcgtcaccgtcgaggaaaacgtcatcgccggcggcgctggcagtgcgatcaacaccttcctgcaggcgcagaaggtgctgatgccggtctgcaacatcggcctgcccgaccgcttcgtcgagcaaggtagtcgcgaggaattgctcagcctggtcggcctcgacagcaagggcatcctcgccaccatcgaacagttttgcgcttaaacttgccgatgctggaaatcattcaactgcc

SEQ ID NO: 53 - DNA, 28 bp, artificial sequence
  primer
atgaaactgaccaccgactatcccttgc

SEQ ID NO: 54 - DNA, 47 bp, artificial sequence
  primer
aatataattgtgtacaagatctggcagttgaatgatttccagcatcg

SEQ ID NO: 55 - DNA, 26 bp, artificial sequence
  primer
tttttttaaggcagttattggtgccc

SEQ ID NO: 56 - DNA, 26 bp, artificial sequence
  primer
tttagcttccttagctcctgaaaatc

SEQ ID NO: 57 - DNA, 1683 bp, artificial sequence
  constructed fragment
gaattcggagcacatcccatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaagtctagttaaagtttaaacgcattctaga

SEQ ID NO: 58 - DNA, 24 bp, artificial sequence
  primer
gttgatcggcacgtaagaggttcc

SEQ ID NO: 59 - DNA, 26 bp, artificial sequence
  primer
tccagcagaacctgattcgcgtcctc

SEQ ID NO: 60 - DNA, 26 bp, artificial sequence
  primer
acatcctggacgaggcgaaggttttc

SEQ ID NO: 61 - DNA, 26 bp, artificial sequence
  primer
acctgccggattacatgaaactgtgc

SEQ ID NO: 62 - DNA, 26 bp, artificial sequence
  primer
aaaaactgggtggtagcctgttcgcg

SEQ ID NO: 63 - DNA, 26 bp, artificial sequence
  primer
gaagtagtcgtcaaaggtcggagtag

SEQ ID NO: 64 - DNA, 26 bp, artificial sequence
  primer
atcagttccagcagggtcagaaattc

SEQ ID NO: 65 - DNA, 25 bp, artificial sequence
  primer
gtaaaccagcaatagacataagcgg

SEQ ID NO: 66 - DNA, 897 bp, artificial sequence
  constructed fragment
gtttaaacttgtggagatcacatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataataatctaga

SEQ ID NO: 67 - DNA, 25 bp, artificial sequence
  primer
ggtttactacatcgtgcattctccg

SEQ ID NO: 68 - DNA, 24 bp, artificial sequence
  primer
gatgagagcagcatgtgttagtcc

SEQ ID NO: 69 - DNA, 264 bp, artificial sequence
  modified Methylomonas sp. 16a hexulose phosphate synthase gene promoter
taaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatccc

SEQ ID NO: 70 - DNA, 475 bp, artificial sequence
  constructed promoter
cctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaagaattcgctcgagtaaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatccc

SEQ ID NO: 71 - DNA, 6325 bp, artificial sequence
  constructed plasmid
ccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaagaattcggagcacatcccatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaagtctagttaaagtttaaacgcattctagaggtttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggaca

SEQ ID NO: 72 - DNA, 6445 bp, artificial sequence
  constructed plsamid
ccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagcgaattctaaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatcacccatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaagtctagttaaagtttaaacgcattctagaggtttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggaca

SEQ ID NO: 73 - DNA, 6584 bp, artificial sequence
  constructed plasmid
ccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaagaattcgctcgagtaaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatcccatggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaaagtctagttaaagtttaaacgcattctagaggtttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggaca

SEQ ID NO: 74 - DNA, 7464 bp, artificial sequence
  constructed plasmid
ccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaagaattcgctcgagtaaggattggggtgcgtcgccggtcgcggcggcgctcctcgacggcagagttggtgccaggttggcggatgattgatgccgaatattacgcgaccaattctcgaggcaaatgaactgtgagctactgagttgcaggcattgacagccatcccatttctatcatacagttacggacgcatcacgagtaggtgataagcctagcagattgcggcagttggcaaaatcagctattactaataattaaaaactttcggagcacatcccatggaggcccgcagaagcgccaactatgagccgaatagctgggactacgactacctgttatcgtccgataccgatgagtccatcgaggtctacaaagacaaagccaaaaagctggaggccgaggttcgccgtgagatcaacaatgagaaggcagagttcttgaccttgctagagttgatagataacgtgcaaaggctcggattgggttatcggttcgagtcagatatccgaggagcactggaccgctttgtctcgtctggaggtttcgatgcagtgaccaaaacgtcgttgcatggcaccgctttgagtttccgtctgttgcgtcaacatggattcgaggtttcacaagaggcgttctccggcttcaaagatcaaaacggtaacttcctggagaacctgaaagaggacatcaaagccattctgagcttgtatgaggcgagctttttggcgctggagggagagaatatcctggatgaggcgaaagtctttgcgatcagtcatctaaaggagttgtcggaggagaaaatcggcaaagagttggcggagcaggtgaaccacgcgttggagctgccgttacaccgcagaacccaacgcctagaggccgtttggtccattgaggcttaccggaaaaaggaggatgccaatcaagtgctgttagagctggccatactggactacaacatgattcagagcgtgtaccaacgcgacttgcgcgagacaagccgctggtggcgtcgagtcggactggccaccaaactgcactttgcccgtgaccgcctgattgagtccttttactgggcagttggcgtcgcgttcgagccccagtacagcgactgccggaatagcgttgcgaaaatgttctgctttgtaaccatcattgacgatatctatgatgtctatggcaccttggatgagttggagctcttcactgacgccgtcgagcgttgggatgtgaatgccatcaatgaccttccagactatatgaagctgtgttttctcgccttgtataacaccatcaacgagatcgcctacgacaacctgaaggataagggcgagaatatcttgccgtacttgaccaaagcttgggctgatctgtgtaacgccttcttgcaggaggcgaaatggttgtataacaaaagtacgcctactttcgacgattacttcggcaacgcatggaaatcgtcatctggaccgctgcaattggtctttgcgtatttcgccgtggtacaaaacatcaaaaaggaggagatagagaatttgcagaagtatcatgacacgatctccaggccatcccatatcttccgcctctgcaatgatcttgcttcggcgagtgccgagattgcccgtggagagaccgccaattctgtgtcatgctatatgcgcaccaagggaatcagcgaggagctggcgaccgagtcggtaatgaaccttattgacgagacctggaaaaagatgaacaaagagaagttgggtgggtcgctgttcgccaaaccctttgtcgagacggctatcaatctggccagacagagccactgcacttaccataacggcgacgcgcacacctcccctgacgagctgacacggaaacgcgtgctgtcggtcattacggagcccatcttaccgtttgagcgctaatagtctagttaaagtttaaacttgtggagatcacatgacagccgacaacaatagcatgccgcatggagccgtcagctcctatgcgaaactggtccaaaatcaaaccccagaggatatcttggaggagttcccggagatcattccgttacaacagcgtccaaacacccggtcgtctgagacgagcaacgacgagtcaggcgagacatgcttcagcggacatgacgaggagcaaatcaaattgatgaacgagaactgcatcgttttagactgggacgataatgcgataggagcgggaaccaaaaaggtttgtcacttgatggagaacattgagaaaggcctgttgcatagggccttcagtgtgttcatctttaacgagcagggcgagctgcttctacaacagcgagccaccgagaagatcaccttcccggacctgtggaccaatacctgctgttcccatccactgtgcatcgacgatgagctgggcctgaaaggaaaactggacgataagatcaagggtgccattactgccgctgtacgtaaactggaccatgagttgggcatcccggaggatgagacgaaaactcgcggaaagtttcacttcttgaatcgcatccactatatggctcccagtaacgagccctggggtgagcacgagattgattacattctgttttacaaaatcaacgcgaaggagaatttgaccgtgaaccccaatgtgaacgaggtccgcgatttcaaatgggtgtcccctaatgatttgaaaacgatgttcgcagatccgtcgtataagtttaccccttggttcaaaatcatttgcgagaactacctgtttaactggtgggagcaattggacgatttgtcggaggtcgagaatgaccgccagatacacagaatgctctaataatctagaggtttttttaaggcagttattggtgcccttaaacgcctggtgctacgcctgaataagtataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcgggacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggaca

(19) United States
(12) Patent Application Publication
(10) Pub. No.: US 2015/0225743 A1
(43) Pub. Date: Aug. 13, 2015

(54) BIOCATALYST FOR CONVERSION OF METHANE AND METHANOL TO ISOPRENE

(71) Applicant: E I Du Pont De Nemours And Company, Wilmington, DE (US)

(72) Inventors: GAIL K. DONALDSON, Newark, DE (US); Kerry Hollands, Newark, DE (US); Stephen K. Picataggio, Rockland, DE (US)

(21) Appl. No.: 14/618,066

(22) Filed: Feb. 10, 2015

Related U.S. Application Data
(60) Provisional application No. 61/937,653, filed on Feb. 10, 2014.

(51) Int. Cl. C12P 5/00; C12N 9/88; C12N 9/90; C12N 15/74

(52) U.S. Cl. CPC C12P 5/007; C12N 15/74; C12N 9/88; C12N 9/90; C12Y 402/03027; C12Y 503/03002

(57) ABSTRACT

Meythylotrophic cells and in particular methanotrophic bacterial cells are genetically engineered to produce isoprene from methane and/or methanol by expressing a heterologous isoprene synthase, and increasing activity of isopentenyl diphosphate isomerase. In addition, upstream DXP pathway enzymes may have increased activity, enzymes in pathways downstream of IPP and DMAPP may have decreased activity, and methane/methanol assimilation pathway enzymes may have increased activity.

This application claims the benefit of U.S. Provisional Application 61/937,653, filed Feb. 10, 2014, and is incorporated by reference in its entirety.

### FIELD OF THE INVENTION

The invention relates to the fields of molecular biology and microbiology. More specifically, biocatalysts and methods for producing isoprene from single carbon substrates are described.

### BACKGROUND OF THE INVENTION

Single carbon substrates provide an abundant, cost effective energy source for a class of microorganisms referred to as methylotrophs, which are characterized by the ability to use carbon substrates lacking carbon to carbon bonds as a sole source of energy. These C1 substrates include methane, methanol, formate, methylated amines and thiols, and various other reduced carbon compounds which lack any carbon-carbon bonds. Although a large number of these organisms are known, few of these microbes have been successfully harnessed to industrial processes for the synthesis of materials.

A subset of methylotrophs are the methanotrophs, which have the ability to utilize methane as a sole carbon source. Methanotrophs convert methane to methanol at ambient temperature and pressure using the enzyme methane monooxygenase. Thus methanotrophs have the potential to harness the abundant, cost effective substrate of methane for production of commercial products.

Methanotrophs are known to accumulate both isoprenoid compounds and carotenoid pigments of various carbon lengths (U.S. Pat. No. 6,660,507; U.S. Pat. No. 6,689,601; Urakami et. al., J. Gen. Appl. Microbiol., 32(4):317-41 (1986)). The methanotroph Methylomonas has been genetically engineered to knockout the native carotenoid pathway of the organism leading to the production of pink-pigmented C₃₀ diapocarotenoids, thereby increasing the available carbon flux directed toward C₄₀ carotenoids of interest, as disclosed in commonly owned U.S. Pat. No. 7,232,666. Genetic engineering of Methylomonas for production of carotenoids has been disclosed in a number of patents including commonly owned U.S. Pat. No. 6,984,523, U.S. Pat. No. 6,969,595, U.S. Pat. No. 7,074,588, U.S. Pat. No. 7,098,000, U.S. Pat. No. 7,252,985, U.S. Pat. No. 7,504,236, U.S. Pat. No. 7,425,625, and U.S. Pat. No. 7,091,031.

Isoprene (2-methyl-1,3-butadiene) is employed in the manufacture of polyisoprene and various copolymers (with isobutylene, butadiene, styrene, or other monomers), most notably used commercially in synthetic rubber for tires. Isoprene can be obtained by direct isolation from petroleum C5 cracking fractions or by dehydration of C5 isoalkanes or isoalkenes (Weissermel and Arpe, Industrial Organic Chemistry, 4^{th} ed., Wiley-VCH, pp. 117-122, 2003). The C5 skeleton can also be synthesized from smaller subunits. It is desirable, however, to have a commercially viable method of producing isoprene from renewable resources.

U.S. Pat. No. 8,470,581 discloses cultured cells expressing a heterologous isoprene synthase that produce isoprene in medium containing uncommon carbon sources such as glycerol, cell mass, protein, alcohol, and plant-derived oil. WO2013063528 discloses variants of a plant isoprene synthase for increased isoprene production in host cells.

There remains a need for methylotrophs and methanotrophs that are genetically engineered for production of isoprene using C1 compounds such as methane as a carbon and energy source.

### SUMMARY OF THE INVENTION

The invention provides recombinant methylotrophic and methanotrophic cells for production of isoprene from the C1 compounds methane and/or methanol, and/or meaning at least one of methane and methanol.

Accordingly, the invention provides recombinant methylotrophic cells comprising:

a) at least one heterologous nucleic acid molecule encoding an isoprene synthase polypeptide;

b) at least one genetic modification which increases isopentenyl diphosphate isomerase activity in the cells as compared with isopentenyl diphosphate isomerase activity in the cells lacking said genetic modification,

wherein the cells produce more isoprene when grown in culture conditions comprising at least one of methanol and methane as carbon source, as compared to the cells without (a) and (b).

In a preferred embodiment the methylotrophic cells are methanotrophic bacterial cells.

In another embodiment the invention provides a method for constructing recombinant methylotrophic cells that produce isoprene comprising:
a) introducing at least one heterologous nucleic acid molecule encoding an isoprene synthase polypeptide; and
b) making at least one genetic modification which increases isopentenyl diphosphate isomerase activity in the cells as compared with isopentenyl diphosphate isomerase activity in the cells lacking said genetic modification.

In another aspect the invention provides a method for production of isoprene comprising:

a) providing a recombinant methylotrophic cell comprising:
i) at least one heterologous nucleic acid molecule encoding an isoprene synthase polypeptide; and
ii) at least one genetic modification which increases isopentenyl diphosphate isomerase activity in the cells as compared with isopentenyl diphosphate isomerase activity in the cell lacking said genetic modification, and

b) growing the cells of (a) on at least one of methane and methanol as carbon source whereby isoprene is produced.

### BRIEF DESCRIPTION OF THE FIGURES AND SEQUENCE DESCRIPTIONS

FIG. 1 shows a diagram of the DXP pathway including Idi, with the addition of IspS to produce isoprene.

FIG. 2 shows a diagram of downstream pathways, following IPP and DMAPP synthesis, leading to production of carotenoids with three arrows representing multiple steps (not necessarily three steps) and multiple enzymes in a line indicating activity of different combinations of these enzymes.

FIG. 3 shows a diagram of methane conversion to formaldehyde in methanotrophic bacteria, including conversion of methanol to formaldehyde.

FIG. 4 shows a diagram of the RuMP cycle.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the nucleotide sequence of Methylomonas sp. 16s 16S rDNA.

SEQ ID NO:2 is the amino acid sequence of the modified P. alba Isps MEA S288C.

SEQ ID NO:3 is the nucleotide sequence of the coding region for the modified P. alba Isps MEA S288C, with E. coli codon optimization.

SEQ ID NO:4 is the nucleotide sequence of the coding region for the modified P. alba Isps MEA S288C, with Methylomonas sp. 16a codon optimization.

SEQ ID NO:5 is the nucleotide sequence of the Pcat promoter from plasmid pC194.

SEQ ID NO:6 is the amino acid sequence of the yeast IDI.

SEQ ID NO:7 is the nucleotide sequence of the native coding region for the yeast IDI.

SEQ ID NO:8 is the nucleotide sequence of the coding region for yeast IDI, with Methylomonas sp. 16a codon optimization.

SEQ ID NO:9 is the amino acid sequence of the coding region for IDI from Pantoea stewartii.

SEQ ID NO:10 is the nucleotide sequence of the coding region for IDI from Pantoea stewartii.

TABLE 1
Coding Region and Polypeptide SEQ ID Numbers

| | SEQ ID NO | SEQ ID NO |
|---|---|---|
| Description | Nucleic acid | amino acid |
| dxs from Methylomonas sp. 16a | 11 | 12 |
| dxr from Methylomonas sp. 16a | 13 | 14 |
| ispD from Methylomonas sp. 16a | 15 | 16 |
| ispE from Methylomonas sp. 16a | 17 | 18 |
| ispF from Methylomonas sp. 16a | 19 | 20 |
| ispG from Methylomonas sp. 16a | 21 | 22 |
| lytB/ispH from Methylomonas sp. 16a | 23 | 24 |
| ispA from Methylomonas sp. 16a | 25 | 26 |
| crtN1 from Methylomonas sp. 16a | 27 | 28 |
| crtN2 from Methylomonas sp. 16a | 29 | 30 |
| crtN3 from Methylomonas sp. 16a | 31 | 32 |
| ald from Methylomonas sp. 16a | 33 | 34 |
| glgA from Methylomonas sp. 16a | 35 | 36 |

SEQ ID NO:37 is the nucleotide sequence of the chromosomal integration vector pGP704/sacBkan-trp.

SEQ ID NO:38 is the nucleotide sequence of the PispFD upstream homologous region fragment.

SEQ ID NOs:39-42, 44, 45, 47, 48, 50, 51, 53-56, 58-65, 67, and 68 are primers.

SEQ ID NO:43 is the nucleotide sequence of the PispFD downstream homologous region fragment.

SEQ ID NO:46 is the nucleotide sequence of the Pdxs1 upstream homologous region fragment,

SEQ ID NO:49 is the nucleotide sequence of the Phps1 promoter fragment.

SEQ ID NO:52 is the nucleotide sequence of the Pdxs1 downstream homologous region fragment.

SEQ ID NO:57 is the nucleotide sequence of the DNA fragment containing an EcoRI site, putative RBS, variant IspS coding region, and PmeI and XbaI restriction sites.

SEQ ID NO:66 is the nucleotide sequence of the DNA fragment containing a PmeI restriction site, putative RBS, idi coding region, and XbaI restriction site.

SEQ ID NO:69 is the nucleotide sequence of the Pₕₚₛ_{_{—}}_{NcoI} promoter.

SEQ ID NO:70 is the nucleotide sequence of the Pcat_Phps promoter.

SEQ ID NO:71 is the nucleotide sequence of the pBHR1-P_{cat}-ispS_S288C plasmid.

SEQ ID NO:72 is the nucleotide sequence of the pBHR1-Pₕₚₛ-ispS_S288C plasmid.

SEQ ID NO:73 is the nucleotide sequence of the pBHR1P_{cat}Pₕₚₛ-ispS_S288C plasmid.

SEQ ID NO:74 is the nucleotide sequence of the pBHR1P_{cat}Pₕₚₛ-ispS_S288C-idi 2.0 plasmid.

### DETAILED DESCRIPTION

The following definitions may be used for the interpretation of the claims and specification:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “methylotroph” means an organism capable of oxidizing organic compounds that do not contain carbon-carbon bonds. Where the methylotroph is able to oxidize CH₄, the methylotroph is also a methanotroph. In one embodiment, the methanotroph uses methanol and/or methane as its primary carbon source.

The term “methanotroph” or “methanotrophic bacteria” means a prokaryote capable of utilizing methane as its primary source of carbon and energy. Complete oxidation of methane to carbon dioxide occurs by aerobic degradation pathways. Typical examples of methanotrophs useful in the present invention include (but are not limited to) the genera Methylomonas, Methylobacter, Methylococcus, and Methylosinus. In one embodiment, the methanotrophic bacteria uses at least one of methane and methanol as its primary carbon source.

The term “gene” refers to a nucleic acid fragment that expresses a specific protein or functional RNA molecule, which may optionally include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”.

The term “expression”, as used herein, refers to the transcription and stable accumulation of coding (mRNA) or functional RNA derived from a gene. Expression may also refer to translation of mRNA into a polypeptide. “Overexpression” refers to the production of a gene product in transgenic organisms that exceeds levels of production in normal or non-transformed organisms.

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “heterologous” means not naturally found in the location of interest. For example, a heterologous gene refers to a gene that is not naturally found in the host organism, but that is introduced into the host organism by gene transfer. For example, a heterologous nucleic acid molecule that is present in a chimeric gene is a nucleic acid molecule that is not naturally found associated with the other segments of the chimeric gene, such as the nucleic acid molecules having the coding region and promoter segments not naturally being associated with each other.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein, an “endogenous nucleic acid” is a nucleic acid whose nucleic acid sequence is naturally found in the host cell.

As used herein, an “endogenous polypeptide” is one that is naturally found in the host cell.

As used herein, the term “Isoprene” refers to 2-methyl-1,3-butadiene (CAS#78-79-5). It can refer to the direct and final volatile C5 hydrocarbon product from the elimination of pyrophosphate from 3,3-dimethylallyl pyrophosphate (DMAPP). It may not involve the linking or polymerization of one or more isopentenyl diphosphate (IPP) molecules to one or more DMAPP molecules. Isoprene is not limited by the method of its manufacture.

As used herein, the terms “isoprene synthase,” “isoprene synthase variant”, and “IspS,” refer to enzymes that catalyze the elimination of pyrophosphate from dimethylallyl diphosphate (DMAPP) to form isoprene. Isoprene synthase enzymes belong to the enzyme classification group EC 4.2.3.27. An “isoprene synthase” may be a wild type sequence or an isoprene synthase variant.

An “isoprene synthase variant” indicates a non-wild type polypeptide having isoprene synthase activity. One skilled in the art can measure isoprene synthase activity using known methods. See, for example, by GC-MS (see, e.g., WO 2009/132220, Example 3 or Silver et al., J. Biol. Chem. 270:13010-13016, 1995). Variants may have one or more of substitutions, additions, deletions, and truncations from a wild type isoprene synthase sequence. Variants may have one or more of substitutions, additions, deletions, and truncations from a non-wild type isoprene synthase sequence. The variants referred to herein may contain at least one amino acid residue substitution from a parent isoprene synthase polypeptide. In some embodiments, the parent isoprene synthase polypeptide is a wild type sequence. In some embodiments, the parent isoprene synthase polypeptide is a non-wild type sequence. In some embodiments, the parent isoprene synthase polypeptide is a naturally occurring sequence.

As used herein, the terms “isopentenyl diphosphate isomerase” and “IDI” refer to an enzyme having activity for conversion of isopentenyl pyrophosphate (IPP) to dimethylallyl pyrophosphate (DMAPP), and for the reverse conversion of DMAPP to IPP. The enzyme belongs to the classification group EC 5.3.3.2.

As used herein, the term “dxs” refers to a gene encoding the enzyme 1-deoxyxylulose-5-phosphate synthase which converts pyruvate and D-glyceraldehyde-3-phosphate into 1-deoxy-D-xylulose-5-phosphate. It belongs to the classification group EC 2.2.1.7.

As used herein, the term “dxr” refers to a gene encoding 1-deoxy-D-xylulose-5-phosphate reductoisomerase which converts 1-deoxy-D-xylulose 5-phosphate to 2-C-methyl-D-erythritol 4-phosphate. It belongs to the classification group EC 1.1.1.267.

As used herein, the term “ispD” refers to a gene encoding a 2C-methyl-D-erythritol cytidyltransferase enzyme which converts 2-C-methyl-D-erythritol-4-phosphate (MEP) and CTP to 4-diphosphocytidyl-2-C-methyl-D-erythritol (CDP-ME). It belongs to the classification group EC 2.7.7.60.

As used herein, the term “ispE” refers to a gene encoding 4-diphosphocytidyl-2-C-methyl-D-erythritol kinase which catalyzes the phosphorylation of the position 2 hydroxy group of 4-diphosphocytidyl-2-C-methyl-D-erythritol. It belongs to the classification group EC 2.7.1.148.

As used herein, the term “ispF” refers to a gene encoding a 2C-methyl-D-erythritol 2,4-cyclodiphosphate synthase which converts 4-diphosphocytidyl-2-C-methyl-D-erythritol 2-phosphate into 2-C-methyl-D-erythritol 2,4-cyclodiphosphate and CMP. It belongs to the classification group EC 4.6.1.12.

As used herein, the term “ispG” refers to a gene encoding 1-hydroxyl-2-methyl-2-(E)-butenyl-4-diphosphate synthase which converts 2-C-methyl-D-erythritol 2,4-cyclodiphosphate to (E)-4-hydroxy-3-methylbut-2-3n-1-yl diphosphate (HMPP).

As used herein, the term “ispH” refers to a gene encoding 4-hydroxy-3-methylbut-2-enyl diphosphate reductase which converts (E)-4-hydroxy-3-methylbut-2-3n-1-yl diphosphate to isopentenyl diphosphate or dimethylallyl diphosphate. It belongs to the classification group EC 1.17.1.2.

The term “ispA” refers to a gene encoding any geranyltransferase or farnesyl diphosphate (FPP) synthase enzyme or any member of the prenyl transferase family of enzymes that can catalyze the condensation of isopentenyl diphosphate (IPP) with 3,3-dimethylallyl diphosphate (DMAPP) or geranyl diphosphate (GPP) to yield FPP in any organism.

The terms “crtN1 gene cluster”, “C₃₀ crt gene cluster”, “crt gene cluster” refer to an operon comprising crtN1, ald, and crtN2 genes that is active in the native carotenoid biosynthetic pathway of Methylomonas sp. 16a.

The term “CrtN1” refers to an enzyme encoded by the crtN1 gene, which is a diapophytoene dehydrogenase or desaturase.

The term “ALD” refers to an enzyme encoded by the ald gene, which is an aldehyde dehydrogenase and is active in the native carotenoid biosynthetic pathway of Methylomonas sp. 16a.

The term “CrtN2” refers to an enzyme encoded by the crtN2 gene, which is a diapophytoene dehydrogenase or desaturase.

The term “CrtN3” refers to an enzyme encoded by the crtN3 gene, whose function is not identified. CrtN3 is active in the native carotenoid biosynthetic pathway of Methylomonas sp. 16a.

The term “Sqs” refers to the squalene synthase enzyme encoded by the sqs gene.

The term “CrtM” refers to an enzyme that performs the head-to-head condensation of two molecules of FPP forming dehydrosqualene as the first committed reaction toward C₃₀ carotenoid biosynthesis, called dehydrosqualene synthase or diapophytoene synthase.

As used herein, the term “glycogen synthase” refers to the enzyme responsible for catalyzing glycogen chain elongation through the addition of adenylated glucose units in the form of ADP-glucose to a glycogen chain (E.C. 2.4.1.21).

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign program of the LASERGENE bioinformatics computing suite (DNASTAR Inc., Madison, Wis.).

Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign v8.0 program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992); Thompson, J. D. et al, Nucleic Acid Research, 22 (22): 4673-4680, 1994) and found in the MegAlign v8.0 program of the LASERGENE bioinformatics computing suite (DNASTAR Inc.). Default parameters for multiple alignment (stated as protein/nucleic acid (GAP PENALTY=10/15, GAP LENGTH PENALTY=0.2/6.66, Delay Divergen Seqs (%)=30/30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 50%, 55%, 60%, 65%, 70%, 75%, 80%, 85%, 90%, or 95%, or any integer percentage from 50% to 100% may be useful in identifying polypeptides of interest, such as 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above identities but typically encode a polypeptide having at least 50 amino acids, preferably at least 100 amino acids, and more preferably at least 125 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et. al., Short Protocols in Molecular Biology, 5^{th} Ed. Current Protocols, John Wiley and Sons, Inc., N.Y., 2002, unless otherwise specified.

The present invention relates to recombinant methylotrophic cells and preferably methanotrophic bacterial cells that synthesize isoprene while utilizing methane or methanol as a carbon source. In the present cells, isoprene is synthesized by the enzyme isoprene synthase (IspS), which is expressed from an introduced heterologous coding region. In addition, isopentenyl diphosphate isomerase activity is increased in the cells to increase the IspS substrate level. Methane provides an abundant, low-cost energy source to support biocatalytic production of isoprene for uses such as in synthetic rubber.

### Isoprene Synthesis in Methylotrophic and Methanotrophic Cells

Host cells useful in the present invention are methylotrophs where the subset of methanotrophs are preferred. All C1 metabolizing microorganisms are generally classified as methylotrophs. Methylotrophs may be defined as any organism capable of oxidizing organic compounds that do not contain carbon-carbon bonds. However, facultative methylotrophs, obligate methylotrophs, and obligate methanotrophs are all various subsets of methylotrophs. Specifically:
Facultative methylotrophs have the ability to oxidize organic compounds which do not contain carbon-carbon bonds, but may also use other carbon substrates such as sugars and complex carbohydrates for energy and biomass;
Obligate methylotrophs are those organisms which are limited to the use of organic compounds that do not contain carbon-carbon bonds for the generation of energy; and
Obligate methanotrophs are those obligate methylotrophs that have the distinct ability to oxidize methane.
   The ability to utilize single carbon substrates is not limited to bacteria but extends also to yeasts and fungi, A number of yeast genera are able to use single carbon substrates in addition to more complex materials as energy sources. Specific methylotrophic yeasts useful in the present invention include but are not limited to Candida, Hansenula, Pichia, Torulopsis, and Rhodotorul. Equivalent species from these genera may be used as hosts herein primarily based upon their demonstrated characterization of being supportable for growth and exploitation on methanol or methane as a single carbon nutrient source. See, for example, Gleeson et al., Yeast 4., 1 (1988).

The ability of obligate methanotrophic bacteria to use methane as their sole source of carbon and energy under ambient conditions, in conjunction with the abundance of methane, makes the biotransformation of methane a potentially unique and valuable process.

In the present cells, at least one heterologous nucleic acid molecule encoding an isoprene synthase polypeptide (IspS) is introduced into methylotrophic host cells, and at least one genetic modification is made in the cells which increases isopentenyl diphosphate isomerase activity. Examples of methanotrophs that may be used include members of the genera Methylomonas, Methylobacter, Methylococcus, and Methylosinus, Methylocyctis, and Methylomicrobium.

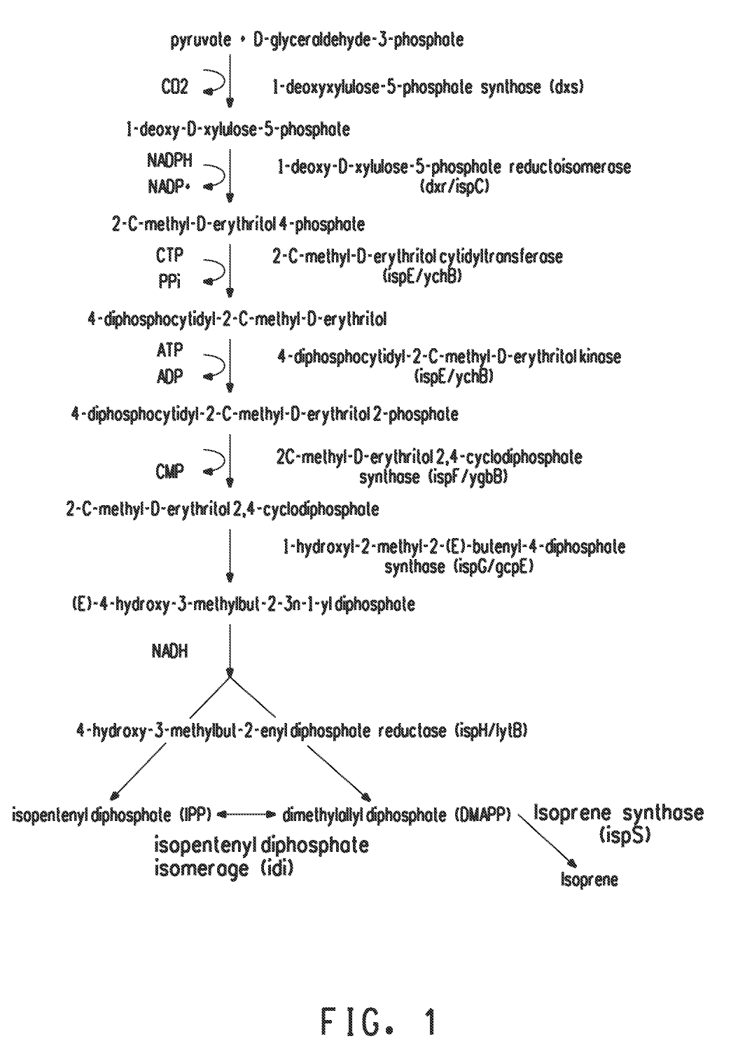

The substrate of isoprene synthase for the synthesis of isoprene is dimethylallyl-diphosphate (DMAPP). DMAPP is made along with isopentenyl diphosphate (IPP) in methanotrophs typically using a pathway for synthesis of terpenoids, isoprenoids and/or terpenes, the DXP (1-deoxy-D-xylulose-5-phosphate) pathway, which is also called the non-mevalonate pathway, mevalonic acid-independent pathway, or MEP (methyl erythritol phosphate) pathway. FIG. 1 shows the DXP pathway including idi, with the addition of ispS. The DXP pathway is characterized by, but not limited to, the enzymes encoded by the following genes: dxs encoding 1-deoxyxylulose-5-phosphate synthase; dxr (also known as ispC) encoding 1-deoxy-D-xylulose-5-phosphate reductoisomerase; ispD (also known as ygbP) encoding a 2C-methyl-D-erythritol cytidyltransferase enzyme; ispE (also known as ychB) encoding 4-diphosphocytidyl-2-C-methyl-D-erythritol kinase; ispF (also known as ygbB) encoding a 2C-methyl-D-erythritol 2,4-cyclodiphosphate synthase; ispG (also known as gcpE) encoding 1-hydroxyl-2-methyl-2-(E)-butenyl-4-diphosphate synthase; ispH (also known as lytB) encoding 4-hydroxy-3-methylbut-2-enyl diphosphate reductase; and idi encoding isopentenyl diphosphate isomerase (IDI).

In the DXP pathway, the product of 1-hydroxyl-2-methyl-2-(E)-butenyl-4-diphosphate synthase is (E)-4-hydroxy-3-methylbut-2-3n-1-yl diphosphate. This product is converted to both isopentenyl diphosphate (IPP) and DMAPP by 4-hydroxy-3-methylbut-2-enyl diphosphate reductase. DMAPP is the substrate for isoprene synthase for synthesis of isoprene. IPP and DMAPP are each substrates for IDI, which interconverts these two compounds. Thus increased activity of IDI provides increased DMAPP substrate for isoprene synthase to produce isoprene.

Methanotrophs are able to produce the initial substrates for the DXP pathway, which are pyruvate and glyceraldehyde-3-phosphate, from methane and/or methanol. Thus any methanotroph having a DXP pathway provides a host cell to be genetically engineered for production of isoprene from at least one of methane and methanol.

In one embodiment the methanotroph has an active Embden-Meyerhof Pathway (which utilizes fructose bisphosphate aldolase) which provides an energetically favorable carbon flux pathway, as disclosed in commonly owned U.S. Pat. No. 7,232,666, which is incorporated herein by reference. Methanotrophs having an active Embden-Meyerhof Pathway include Methylomonas clara, Methylosinus sporium, and Methylomonas sp. 16a. A particular feature of the Embden-Meyerhof pathway in Methylomonas sp. 16a is that the key phosphofructokinase step is pyrophosphate-dependent instead of ATP-dependent. This feature adds to the energy yield of the pathway by using pyrophosphate instead of ATP, making Methylomonas sp. 16a a particularly desirable host strain due to its high growth as disclosed in U.S. Pat. No. 6,689,601, which is incorporated herein by reference. Methylomonas sp. 16a (ATCC PTA-2402) and strains derived therefrom may be identified using the 16S rDNA sequence of SEQ ID NO:1.

### IspS and IDI Expression

In the present host cells, any heterologous nucleic acid molecule encoding a polypeptide with isoprene synthase activity may be expressed for synthesis of isoprene using at least one of methane and methanol as a carbon source. One or more heterologous nucleic acid molecule encoding a polypeptide with isoprene synthase activity may be expressed. Isoprene synthase enzymes belong to the enzyme classification group EC 4.2.3.27. Standard methods can be used to determine whether a polypeptide has isoprene synthase activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one aspect, DMAPP (Sigma) can be evaporated to dryness under a stream of nitrogen and rehydrated to a concentration of 100 mM in 100 mM potassium phosphate buffer pH 8.2 and stored at −20° C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml headspace vial with a metal screw cap and Teflon® coated silicon septum (Agilent Technologies) and cultured at 37° C. for 15 minutes with shaking. The reaction can be quenched by adding 200 μL of 250 mM EDTA and quantified by GC/MS.

Polypeptides with isoprene synthase activity may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis using for example the sequence of a modified P. alba isoprene synthase (SEQ ID NO:2).

In one embodiment the heterologous nucleic acid molecule encoding IspS may comprise a wild-type or natural IspS encoding sequence. In other embodiments the heterologous nucleic acid molecule encoding IspS may be a variant that encodes a variant polypeptide as disclosed in WO2013063528, which is incorporated herein by reference, such as having one or more of substitutions, additions, deletions, and truncations. A variant IspS may have improved expression, stability, solubility and/or activity as disclosed therein. Variants that may be used include those disclosed in US20130164808, which is incorporated herein by reference. In one embodiment the IspS variant has a truncation at the N-terminus, for example the truncated P. alba IspS of SEQ ID NO:2. The amino acid sequence of SEQ ID NO:2 in addition is a variant having a substitution of cysteine for serine at position 288 of SEQ ID NO:2. The coding sequence for the amino acid sequence of SEQ ID NO:2, which is a codon optimized variant of the native P. alba coding sequence for expression in E. coli, is SEQ ID NO:3.

In some embodiments the expressed IspS is less susceptible to degradation, such as degradation by proteases, as disclosed in WO2013181647, which is incorporated herein by reference. In some aspects, the isoprene synthase polypeptide (e.g., a variant) has one or more substitutions in the wild-type or naturally occurring isoprene synthase polypeptide, wherein the isoprene synthase polypeptide is more resistant to degradation by protease(s). In some aspects, the degradation of isoprene synthase polypeptide in the cells when using such isoprene synthase polypeptide is less compared to the degradation of isoprene synthase polypeptide in the cells when using a wild-type or naturally occurring isoprene synthase.

In some embodiments the coding region for IspS encodes a plant isoprene synthase polypeptide or a variant thereof. The IspS coding region may be a native or variant sequence isolated from a plant species selected from poplar (Populus sp.), kudzu (Pueraria sp.), English oak (Quercus sp.) or willow (Salix sp.). In another embodiment, the parent species is Populus sp. In another embodiment, in the parent is P. alba, P. tremuloides, P. trichocharpa, P. nigra or a Populus hybrid such as Populus alba×Populus tremula. In another embodiment, the parent species is Pueraria sp. In another embodiment, in the parent species is Pueraria montana. In another embodiment, the parent species is Quercus sp. In another embodiment, the parent species is Quercus rubur. In another embodiment, the parent species is Salix sp. In another embodiment, the parent species is S. alba or S. baylonica.

In some aspects, the nucleic acid encoding IspS is codon optimized, for example, codon optimized based on host cells where the heterologous isoprene synthase is to be expressed. For example, the nucleic acid encoding a variant of isoprene synthase from Populus alba may be codon optimized for expression in E. coli (SEQ ID NO:3) or in a methanotroph such as Methylomonas (SEQ ID NO:4).

The isoprene synthase polypeptide encoded by the heterologous nucleic acid molecule described herein may be any of the isoprene synthases or isoprene synthase variants described in WO 2009/132220, WO 2010/124146, and U.S. Patent Application Publication No.: 2010/0086978, U.S. Pat. No. 8,173,410, and U.S. patent application Ser. No. 13/283,564 (US 2013/0045891), the contents of each of which are incorporated herein by reference in their entirety with respect to the isoprene synthases and isoprene synthase variants.

Suitable isoprene synthases include, but are not limited to, those identified by Genbank Accession Nos. AY341431, AY316691, AY279379, AJ457070, and AY182241. Types of isoprene synthases which can be used in any one of the compositions and methods described herein are also described in International Patent Application Publication Nos. WO2009/076676, WO2010/003007, WO2009/132220, WO2010/031062, WO2010/031068, WO2010/031076, WO2010/013077, WO2010/031079, W02010/148/150, WO2010/124146, WO2010/078457, WO2010/148256, and Sharkey et al., Evolution (2012) (available on line at DOI: 10.1111/evo.12013), the contents of each of which are incorporated herein by reference.

A nucleic acid molecule encoding any of the IspS polypeptides described above and in the references given above is operably linked to a promoter that is active in the cells chosen for expression as the host cells, in a chimeric gene. Promoters may be constitutive or inducible. Examples of promoters useful for expression in Methylomonas cells include a chloramphenicol resistance gene promoter (Pcat; SEQ ID NO: 5) from plasmid pC194 (Horinouchi and Weisblum, J Bacteriol. (1982) 150:815-825), a Phps1 promoter from the Methylomonas hexulose phosphate synthase gene, and the inducible promoters disclosed in U.S. Pat. No. 7,098,005, which is incorporated herein by reference. Typically a transcription termination sequence is also included in a chimeric gene constructed for expression in the desired host cells. Vectors to carry chimeric genes and transformation methods are well known by those skilled in the art. Chimeric genes for expression, such as one encoding IspS, may be integrated into the genome of the host cells or can be stably maintained on a vector in the cells.

In the present recombinant methylotrophic host cells, at least one genetic modification is made which increases isopentenyl diphosphate isomerase (IDI) activity in the cells. Methanotrophic bacterial host cells may or may not have endogenous IDI activity. Thus increased activity may be higher activity than an activity level that is already present in the host cells (endogenous activity). Alternatively, increased activity may be activity in host cells which do not have endogenous IDI activity, where the activity is increased from no activity to a detectable level of activity.

Any nucleic acid molecule encoding an isopentenyl diphosphate isomerase enzyme may be used in the genetic modification. The isopentenyl diphosphate isomerase enzyme belongs to the enzyme classification group EC 5.3.3.2. Standard methods can be used to determine whether a polypeptide has IDI activity by measuring the ability of the polypeptide to interconvert IPP and DMAPP in vitro, in a cell extract, or in vivo. Various IDI enzymes are described in Berthelot et al. (Biochemie (2102) 94:1621-1634), any of which may be used in the present cells. Polypeptides with isopentenyl diphosphate isomerase activity may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis using for example the sequence of an isopentenyl diphosphate isomerase from S. cerevisiae (SEQ ID NO:6; coding sequence of SEQ ID NO:7) or from Pantoea stewartii (SEQ ID NO:9, coding sequence of SEQ ID NO:10).

Increased expression of isopentenyl diphosphate isomerase may be achieved by any method known to one skilled in the art such as by increasing expression of an endogenous coding region, introducing more copies of an endogenous gene or an endogenous coding region operably linked to a promoter heterologous to the coding region, or introducing a heterologous coding region that is operably linked to a promoter active in host cells for expression. Expression of an endogenous coding region may be increased such as by substituting a more highly active promoter for the endogenous gene promoter.

An IDI used for increased expression in the host cells may be endogenous to the host cells or from another organism. The DXP pathway typically includes isopentenyl diphosphate isomerase which interconverts IPP and DMAPP. Thus a genetic modification may be made that increases expression of an IDI that is endogenous to the host cells. Alternatively, a heterologous IDI may be expressed in the host cells. An IDI may be expressed in the host cell that is from a different methanotroph, a non-methanotroph bacteria, or other organism including yeast. When expressing a heterologous IDI, the encoding sequence may be codonoptimized for expression in the host cells. For example, the yeast IDI (SEQ ID NO:6) may be expressed from the native coding sequence (SEQ ID NO:7), or using coding sequence that is codon optimized for expression in Methylomonas (SEQ ID NO:8).

A nucleic acid molecule encoding any of the IDI polypeptides described above is operably linked to a promoter that is active in the methylotrophic cells chosen for expression as the host cells. Promoters may be as described above for expressing an IspS coding region. When more than one nucleic acid encoding a polypeptide is introduced into a cell, the nucleic acids may be operably linked to separate a promoters, or operably linked in an operon to the same promoter. For example, coding regions for IspS and IDI may be in one operon expressed from the same promoter. Typically a ribosome binding site is included for each coding region upstream of the coding region adjacent to the promoter.

### Additional DXP Pathway Modifications

In various embodiments, at least one genetic modification is made to increase expression of at least one gene of the DXP pathway, shown in FIG. 1 (the native DXP pathway does not include ispS, and may or may not include idi), in addition to increased isopentenyl diphosphate isomerase expression, wherein expression is higher than in cells without the modification. Any combination of genes of the pathway may be increased in expression. Genes that may have increased expression include those encoding 1-deoxyxylulose-5-phosphate synthase (dxs), 1-deoxy-D-xylulose-5-phosphate reductoisomerase (dxr or ispC), 2C-methyl-D-erythritol cytidyltransferase (ispD or ygbP), 4-diphosphocytidyl-2-C-methyl-D-erythritol kinase (ispE or ychB), 2C-methyl-D-erythritol 2,4-cyclodiphosphate synthase (ispF or ygbB), 1-hydroxyl-2-methyl-2-(E)-butenyl-4-diphosphate synthase ispG or gcpE), and 4-hydroxy-3-methylbut-2-enyl diphosphate reductase (ispH or lytB). Examples of these polypeptides are their amino acid sequences from Methylomonas 16a which are SEQ ID NOs:12, 14, 16, 18, 20, 22, and 24, respectively. Coding sequences for these polypeptides are SEQ ID NOs:11, 13, 15, 17, 19, 21, and 23, respectively.

Polypeptides having any of these activities may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis using for example the sequences of SEQ ID NOs: 12, 14, 16, 18, 20, 22, and 24.

Increased expression of any of these enzymes may be achieved by any method known to one skilled in the art. Either an endogenous coding region or a heterologous coding region for the enzyme to be increased in expression may be used. Methods for increasing expression include increasing expression of an endogenous coding region, introducing more copies of an endogenous gene or an endogenous coding region operably linked to a promoter heterologous to the coding region, or introducing a heterologous coding region that is operably linked to a promoter active in the host cells. Expression of an endogenous coding region may be increased such as by substituting a more highly active promoter for the endogenous gene promoter or regulatory region. Any heterologous coding region to be expressed may be codon optimized for the host cell.

1-deoxyxylulose-5-phosphate synthase is encoded by dxs and converts pyruvate and D-glyceraldehyde-3-phosphate into 1-deoxy-D-xylulose-5-phosphate. It belongs to the classification group EC 2.2.1.7. For example, 1-deoxyxylulose-5-phosphate synthase expression is increased herein by substituting a more highly active promoter, the Phps1 promoter from the Methylomonas hexulose phosphate synthase gene, for the endogenous promoter in Methylomonas sp. 16a.

2-C-methyl-D-erythritol cytidyltransferase enzyme is encoded by ispD (also called ygbP) and converts 2-C-methyl-D-erythritol-4-phosphate (MEP) and CTP to 4-diphosphocytidyl-2-C-methyl-D-erythritol (CDP-ME). It belongs to the classification group EC 2.7.7.60. 2C-methyl-D-erythritol 2,4-cyclodiphosphate (HMBPP) synthase is encoded by ispF (also called ygbB) and converts 4-diphosphocytidyl-2-C-methyl-D-erythritol 2-phosphate into 2-C-methyl-D-erythritol 2,4-cyclodiphosphate and CMP. It belongs to the classification group EC 4.6.1.12. For example, expression of these two enzymes is increased herein in Methylomonas sp. 16a cells by substituting a chloramphenicol resistance gene promoter (Pcat) (SEQ ID NO:5) from plasmid pC194 (Horinouchi and Weisblum, J Bacteriol. (1982) 150:815-825), for the endogenous promoter of the ispFD operon in Methylomonas 16a.

Any other enzyme of the DXP pathway, either alone or in any combination, may be increased in expression in the present cells. 1-deoxy-D-xylulose-5-phosphate reductoisomerase is encoded by dxr (also called ispC) and converts 1-deoxy-D-xylulose 5-phosphate to 2-C-methyl-D-erythritol 4-phosphate. It belongs to the classification group EC 1.1.1.267. 4-diphosphocytidyl-2-C-methyl-D-erythritol kinase is encoded by ispE (also ychB) and catalyzes the phosphorylation of the position 2 hydroxy group of 4-diphosphocytidyl-2-C-methyl-D-erythritol. It belongs to the classification group EC 2.7.1.148. 1-hydroxyl-2-methyl-2-(E)-butenyl-4-diphosphate synthase is encoded by ispG (also gcpE) and converts 2-C-methyl-D-erythritol 2,4-cyclodiphosphate to (E)-4-hydroxy-3-methylbut-2-3n-1-yl diphosphate (HMPP). HMBPP reductase (also 4-hydroxy-3-methylbut-2-enyl diphosphate reductase) is encoded by ispH (also lytB) and converts (E)-4-hydroxy-3-methylbut-2-3n-1-yl diphosphate to isopentenyl diphosphate or dimethylallyl diphosphate. It belongs to the classification group EC 1.17.1.2.

Examples of genes encoding enzymes of the DXP pathway are given by accession number in Table 2 of U.S. Pat. No. 6,969,595, which is incorporated herein by reference. Coding sequences for DXP pathway enzymes from Methylomonas sp. 16a may be used, such as those from the genes dxs: SEQ ID NO:11 (protein SEQ ID NO:12); dxr: SEQ ID NO:13 (protein SEQ ID NO:14), ispD: SEQ ID NO:15 (protein SEQ ID NO:16), ispE: SEQ ID NO:17 (protein SEQ ID NO:18), ispF: SEQ ID NO:19 (protein SEQ ID NO:20), ispG: SEQ ID NO:21 (protein SEQ ID NO:22), lytB/ispH: SEQ ID NO:23 (protein SEQ ID NO:24).

### Competing Pathway Modifications

In various embodiments, at least one genetic modification is made to decrease expression of one or more endogenous gene encoding an enzyme in a pathway downstream of IPP and DMAPP. A pathway downstream of IPP and DMAPP uses one or both of IPP and DMAPP as substrate in a first enzymatic step of the pathway. Decreased carbon flow through IPP and DMAPP to other products provides more substrate for isoprene synthase. Downstream pathways may include pathways for synthesis of monoterpenes from geranyl-PP, sesquiterpenes and triterpenes from farnesyl-PP, and diterpenes and tetraterpenes from geranylgeranyl-PP. Downstream enzymes include dimethylallyltranstransferase classified as EC 2.5.1.1 which converts dimethylallyl diphosphate and isopentenyl diphosphate to diphosphate and geranyl diphosphate; (2E,6E)-farnesyl diphosphate synthase classified as EC 2.5.1.10 which interconverts geranyl diphosphate and isopentenyl diphosphate with diphosphate and (2E,6E)-farnesyl diphosphate; and geranylgeranyl diphosphate synthase classified as EC 2.5.1.29 which converts (2E,6E)-farnesyl diphosphate+isopentenyl diphosphate to diphosphate+geranylgeranyl diphosphate,

Polypeptides having any of these activities may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis using, for example, the SEQ ID NO:26 for IspA.

Expression of any combination of downstream pathway genes may be decreased, which includes either reducing gene expression or eliminating gene expression. Expression is reduced or eliminated by any method known to one skilled in the art. For example, expression may be reduced or eliminated by insertion into the coding region whereby the protein is truncated or mutated, or into the promoter whereby the promoter activity is reduced or eliminated. Also a weak promoter may be substituted for the native promoter.

In one embodiment expression of IspA is decreased but not eliminated as disclosed in US20130164808. The IspA polypeptide is any geranyltransferase or farnesyl diphosphate (FPP) synthase enzyme or any member of the prenyl transferase family of enzymes that can catalyze a sequence of two prenyltransferase reactions leading to the creation of geranyl pyrophosphate (GPP; a 10-carbon molecule) and farnesyl pyrophosphate (FPP; a 15-carbon molecule). In some embodiments, IspA is encoded by an ispA gene. For example, the ispA coding sequence from Methylomonas sp. 16a is SEQ ID NO:25, and the IspA polypeptide is SEQ ID NO:26.

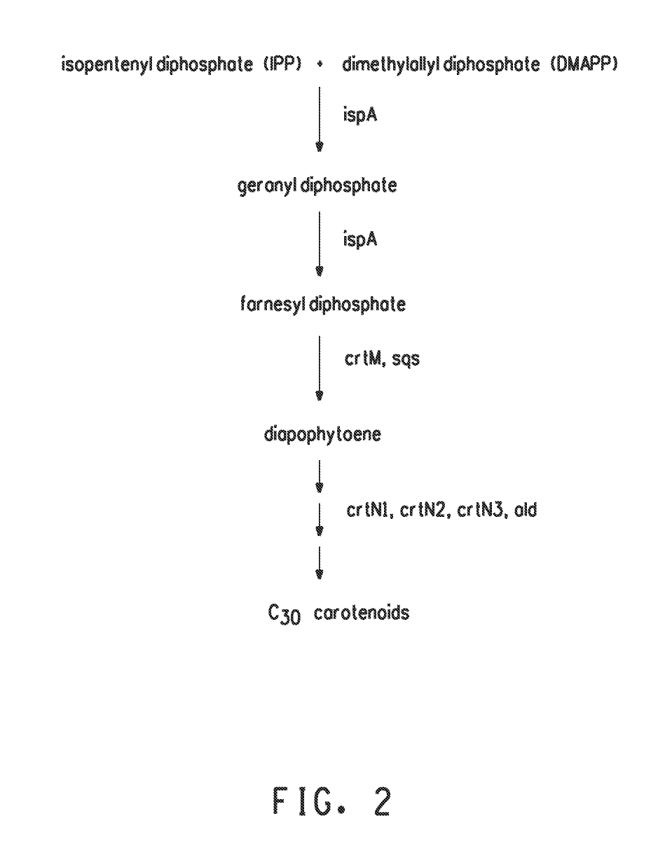

In various embodiments the host methanotroph cells synthesize carotenoids using pathways that are downstream of the pathway shown in FIG. 1 producing IPP and DMAPP, such as the general pathway for synthesis of C₃₀ carotenoids shown in FIG. 2. Examples of methanotrophic bacterial cells making carotenoids using at least some of the described enzymes include Methylomonas methanica, Methylomonas fodinarum, Methylomonas aurantiaca, and Methylomonas 16a (ATCC PTA-2402; disclosed in U.S. Pat. No. 6,689,601, which is incorporated herein by reference). In various embodiments one or more genes for production of carotenoids is decreased, such as genes in the pathway shown in FIG. 2. Different C₃₀ carotenoids are made in the C₃₀ carotenoid pathway using different combinations of enzymes that may include the enzymes encoded by the following genes: sqs encoding squalene synthase, crtM encoding an enzyme that performs the head-to-head condensation of two molecules of FPP forming dehydrosqualene, crtN1 encoding diapophytoene dehydrogenase or desaturase, crtN2 encoding diapophytoene dehydrogenase or desaturase, crtN3 (whose encoded enzyme is not identified), and ald encoding a putative aldehyde dehydrogenase. C₃₀ carotenoid production may be decreased in a methanotroph that produces C₃₀ catrotenoids as disclosed in U.S. Pat. No. 7,323,666, which is incorporated herein by reference. For example, the genes ald, crtN1, crtN2, and crtN3 were disrupted in Methylomonas sp. 16a, thereby eliminating production of C₃₀ carotenoids creating white strains.

Some examples of sequences in the C₃₀ carotenoid pathway are those from Methylomonas sp. 16a: crtN1 coding sequence of SEQ ID NO:27, polypeptide SEQ ID NO:28; crtN2 coding sequence of SEQ ID NO:29, polypeptide SEQ ID NO:30; crtN3 coding sequence of SEQ ID NO:31, polypeptide SEQ ID NO:32; and ald coding sequence of SEQ ID NO:33, polypeptide SEQ ID NO:34.

In addition, other competing pathways may be reduced in activity. For example, expression of glycogen synthase may be reduced as disclosed in U.S. Pat. No. 7,217,537 and U.S. Pat. No. 7,504,236, which are incorporated herein by reference, wherein the endogenous glgA gene encoding glycogen synthase is disrupted or deleted. Glycogen synthase (E.C. 2.4.1.21), encoded by the gene glgA, is responsible for catalyzing glycogen chain elongation through the addition of adenylated glucose units in the form of ADP-glucose to a glycogen chain. Glycogen is the main carbon and energy storage product in most animals, fungi, algae, and bacteria. An example of a glycogen synthase is that from Methylomonas sp. 16a: glgA coding sequence of SEQ ID NO:35, polypeptide SEQ ID NO:36. Polypeptides having any of these activities to be decreased may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis using for example SEQ ID NOs:25, 27, 29, 31, 33, and 35.

### Upstream Pathway Modifications

Activities of one or more enzymes involved in at least one of methane and methanol assimilation in a methanotrophic bacterial host cell may be increased to provide more carbon flow to the DXP pathway for isoprene production.

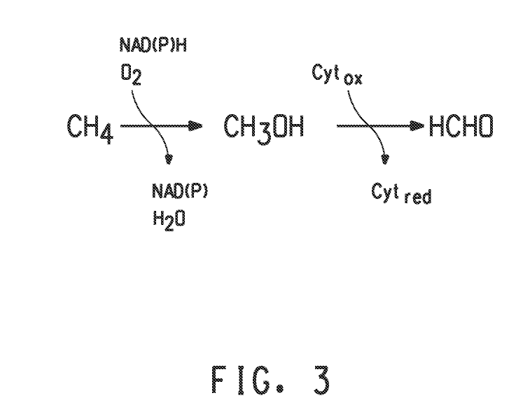

Methanotrophic bacteria use the enzyme methane monooxygenase (MMO; EC 1.14.13.25) to catalyze the oxidation of methane to methanol. Methane monooxygenase is oxygen-dependent and requires reducing equivalents to activate the oxygen. The oxygen molecule is split during catalysis, with one atom reduced to water and the other incorporated into methane to produce methanol. All methanotrophs express a membrane-bound particulate MMO (pMMO), which contains copper and apparently uses a quinol as the electron donor. A few species of methanotrophic bacteria, including Methylomonas, produce a second, soluble form of MMO (sMMO) that does not contain copper. sMMO is a cytosolic multi-component enzyme that utilizes NAD(P)H as the electron donor (see FIG. 3). The hydroxylase component of the enzyme contains two dinuclear iron active sites. Differential expression of the two enzyme systems is regulated by the availability of copper ions. When the copper to biomass ratio is low, sMMO activity is observed, whereas pMMO is expressed at high copper to biomass ratios.

Methanol from both endogenous and exogenous sources is subsequently oxidized to formaldehyde (see FIG. 3) by a periplasmic methanol dehydrogenase (MDH: EC 1.1.2.7). Electrons are transferred from MDH to an atypical cytochrome cL, which in turn is oxidized by a typical class I cytochrome c (cH). The two cytochromes are periplasmic as well.

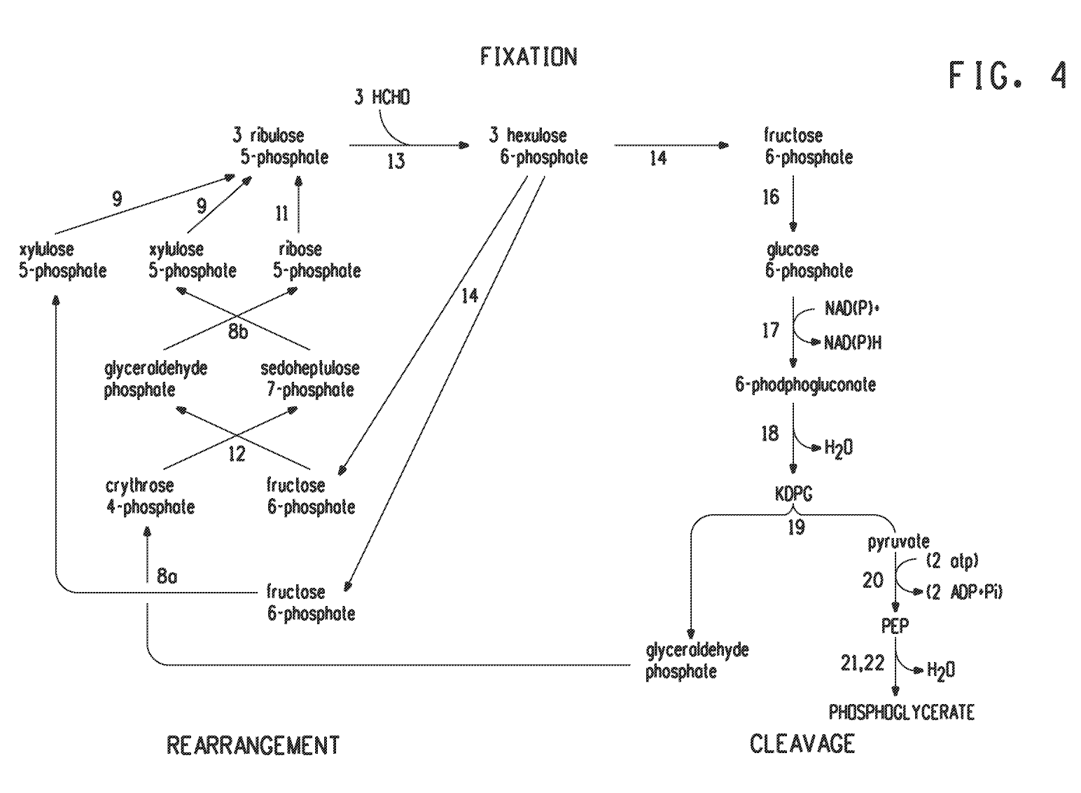

Formaldehyde produced from the oxidation of methane and methanol is assimilated to form metabolic intermediates that are subsequently used for biosynthesis of cell material. There are two known pathways used by methylotrophic bacteria for the synthesis of multicarbon compounds from formaldehyde. In the serine pathway, 2 molecules of formaldehyde and 1 molecule of carbon dioxide are utilized in each cycle forming a three-carbon intermediate, while in the RuMP cycle, 3 molecules of formaldehyde are assimilated forming a three-carbon intermediate of central metabolism (see FIG. 4). In the latter pathway, all cellular carbon is assimilated at the oxidation level of formaldehyde.

The RuMP pathway consists of three parts—fixation, cleavage and rearrangement. In the fixation part of the cycle, formaldehyde and D-ribulose 5-phosphate (RuMP) are condensed by hexulose-6-phosphate synthase (HPS; EC 4.1.2.43) to form hexulose 6-phosphate (HuMP), which in turn is converted to D-fructose 6-phosphate (FMP) by 6-phospho-3-hexuloisomerase (HPI; EC 5.3.1.27). These two enzymes are the only enzymes which are unique to organisms that employ this pathway. Three molecules of FMP are produced from the assimilation of three molecules of formaldehyde. The enzymes which participate in the other two parts of the RuMP pathway are members of other pathways, such as the non-oxidative branch of pentose phosphate pathway, and are not unique to organisms utilizing the RuMP pathway.

In the cleavage part of the RuMP pathway, some of the FMP is cleaved to 3-carbon compounds. There are two possible routes: in the first route FMP is phosphorylated by 6-phosphofructokinase (EC 2.7.1.11) to fructose 1,6-bisphosphate (FDP), which is then cleaved by fructose-bisphosphate aldolase (EC 4.1.2.13) to dihydroxy acetone phosphate (DHAP) and glyceraldehyde 3-phosphate. In the second route FMP is first isomerized to glucose 6-phosphate (GMP) by glucose-6-phosphate isomerase (EC 5.3.1.9). GMP is dehydrogenated first to D-glucono-1,5-lactone 6-phosphate by glucose-6-phosphate 1-dehydrogenase (EC 1.1.1.49), and then to 6-phospho-gluconate by 6-phosphogluconolactonase (EC 3.1.1.31). 6-phospho-gluconate is then converted by two key enzymes of the Entner-Douderoff pathway to 2-keto-3-deoxy-6-phospho-D-gluconate (KDPG) by phosphogluconate dehydratase (EC 4.2.1.12), and KDPG is cleaved into glyceraldehyde 3-phosphate and pyruvate by KDPG aldolase (EC 4.1.2.14). The glyceraldehyde 3-phosphate and pyruvate formed are the two key substrates for the deoxy-xylulose phosphate pathway leading to the synthesis of isoprenoids and carotenoids. The pyruvate or DHAP which are formed are then channeled to biosynthesis. For every three molecules of formaldehyde that are condensed, one molecule of FMP is cleaved, and thus one molecule of pyruvate or DHAP is routed to biosynthesis.

In the final rearrangement step, the RuMP pathway molecules are regenerated by several possible routes. For example, the GAP which is left from the cleaved FMP can react with one of the other two FMP molecules to form xylulose-5-phosphate (XuMP) and erythrose-4-phosphate (EMP). This reaction is catalyzed by a transketolase (EC 2.2.1.1). EMP then reacts with the third FMP to form septulose-7-phosphate (SMP) and glyceraldehyde 3-phosphate (GAP). This reaction is catalyzed by a transaldolase (EC 2.2.1.2). These two compounds are the substrate for another aldolase (also 2.2.1.2), which generates XuMP and a ribose-5-phosphate (RiMP). The net result of these rearrangements reactions are two XuMP and a RiMP, all of which are then converted back to RuMP by ribulose-phosphate 3-epimerase (EC 5.1.3.1) and ribose-5-phosphate isomerase (EC 5.3.1.6), thus closing the cycle.

Any of the described enzymes in the methane/methanol assimilation pathways may be increased in expression in the present cells. Any combination of genes of the pathways may be increased in expression. Polypeptides having any of these activities may be identified using bioinformatics and/or experimental methods. Amino acid sequences of these polypeptides can be readily found by EC number, gene name, and/or enzyme name using databases that are well known to one of skill in the art including NCBI (National Center for Biotechnology Information; Bethesda, Md.), BRENDA (The Comprehensive Enzyme Information System; Technical University of Braunschweig Dept. of Bioinformatics), and Swiss-Prot (Swiss Institute of Bioinformatics; Lausanne, Switzerland). In addition, amino acid sequences of these polypeptides can be readily found based on a known sequence using bioinformatics, including sequence analysis software such as BLAST sequence analysis.

### Industrial Production Methodologies

The present cells are grown using medium comprising at least one of methanol and methane. The gas phase of the culture typically contains about 5 to 50% of methane as a carbon source. Typically salts are present in the liquid phase, for example as given for BTZ medium in Examples herein, with either ammonia or nitrate as a nitrogen source. Under these growth conditions isoprene is produced by the present cells.

For commercial production of the desired product, e.g., isoprene, a variety of culture methodologies may be applied. For example, large-scale production of isoprene from the present Methylomonas sp. 16a bacterial host organism may be by batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur while adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, 2nd ed. (1989) Sinauer Associates: Sunderland, Mass., or Deshpande, Mukund V., Appl. Biochem. Biotechnol., 36:227 (1992).

Commercial production of the desired product, e.g., isoprene, may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock, supra.

Fermentation media in the present invention must contain suitable carbon substrates. Suitable carbon substrates for the present methanotrophic bacterial cells include methane and methanol.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “kb” means kilobase(s), “bp” means base pairs, “nt” means nucleotide(s), “hr” or “h” means hour(s), “min” means minute(s), “sec” means second(s), “d” means day(s), “L” means liter(s), “ml” or “mL” means milliliter(s), “μL” means microliter(s), “μg” means microgram(s), “g” means gram(s), “mg” means milligram(s), “mM” means millimolar, “μM” means micromolar, “M” means molar, “WT” means wild type, “OD₆₀₀” means optical density at 600 nm, “RBS” means ribosome binding site.

### General Methods

### Methylomonas Strains

Methylomonas sp. 16a is a Methylomonas strain with ATCC #PTA-2402.

### Methylomonas Strain MWM1900

Construction of the MWM1900 strain, which is disrupted for production of C₃₀ carotenoids, was described in U.S. Pat. No. 7,232,666 Examples 3, 4, 5, and 7, which are incorporated herein by reference. To make this strain, disruptions of the crt cluster and of crtN3 were made in Methylomonas sp. 16a as described therein. The crt cluster includes the ald, crtN1, and crtN2 coding regions expressed from a single promoter.

### Methylomonas Strain DWS1044

To construct strain DWS1044, the endogenous promoters of the ispFD and dxs1 genes were replaced in Methylomonas strain MWM1200. MWM1200 is a strain of Methylomonas sp. 16a with disruptions in the crt cluster promoter and in crtN3, that was described in U.S. Pat. No. 7,232,666 Examples 3, 4, 5, and 7, which is incorporated herein by reference. Expression of ald, crtN1, and crtN2 is disrupted by disruption of the crt cluster promoter. MWM1200 is disrupted for production of C₃₀ carotenoids.

In MWM1200, the promoter of ispFD was replaced with a chloramphenicol resistance gene promoter (Pcat) (SEQ ID NO:5) from plasmid pC194 (Horinouchi and Weisblum, J Bacteriol. (1982) 150:815-825). Chromosomal ispFD promoter replacement in MWM1200 was made by double-crossover homologous recombination procedure using the chromosomal integration vector pGP704/sacBkan-trp (SEQ ID NO: 37) cloned with a DNA cassette containing a PispFD upstream homologous region, Pcat promoter, and a PispFD downstream homologous region. Plasmid pGP704/sacBkan-trp was derived from pGP704 (Miller and Mekalanos, J. Bacteriol. (1988) 170:2575-2583) and constructed to contain a Bacillus amyloliquifaciens sacB gene under the control of the neutral protease (npr) promoter and a kanamycin resistance gene with a trp terminator in pGP704.

The PispFD upstream homologous region fragment (SEQ ID NO:38) was amplified from Methylomonas sp. 16a genomic DNA with primer ST-ispFD(SpeIXbaI) (SEQ ID NO:39) containing an XbaI restriction enzyme site and primer SB-ispFD(upstream) (SEQ ID NO:40). A Pcat promoter fragment was amplified from plasmid pC194 with primer ST-Pcat(ispFD) (SEQ ID NO:41) and primer SB-Pcat(ispFD) (SEQ ID NO:42). The PispFD downstream homologous region fragment (SEQ ID NO:43) was amplified from Methylomonas sp. 16a genomic DNA with primer ST-ispFD(16a) (SEQ ID NO:44) and primer SB-ispFD(BglIIAcc651) (SEQ ID NO:45) containing a BglII restriction enzyme site. The DNA cassette PispFDupstream-Pcat-PispFDdownstream was made by SOEing PCR among the PispFD upstream homologous fragment, Pcat promoter fragment, and PispFD downstream homologous fragment. The cassette PispFDupstream-Pcat-PispFDdownstream was digested with XbaI and BglII, and cloned in Xba1/BglII sites of pGP704/sacBkan-trp to create pGP704/sacBkan-trp-Pcat-ispFD.

Replacement of the dxs1 promoter was similarly made using the chromosomal integration vector pGP704/sacBkan-trp (SEQ ID NO:37) cloned with a DNA cassette containing a Pdxs1 upstream homologous region, Phps1 promoter (from the Methylomonas hexulose phosphate synthase gene), and a Pdxs1 downstream homologous region.

The Pdxs1 upstream homologous region fragment (SEQ ID NO:46) was amplified from Methylomonas sp. 16a genomic DNA with primer ST-dxs1(NheIXbaI) (SEQ ID NO:47) containing an XbaI restriction enzyme site and primer SB-dxs1 (upstream) (SEQ ID NO:48). The Phps1 promoter fragment (SEQ ID NO:49) was amplified from Methylomonas sp. 16a genomic DNA with primer ST-Phps1(dxs1) (SEQ ID NO:50) and primer SB-Phps1(dxs1) (SEQ ID NO:51). The Pdxs1 downstream homologous region fragment (SEQ ID NO:52) was amplified from Methylomonas 16a genomic DNA with primer ST-dxs1 (16a) (SEQ ID NO:53) and primer SB-dxs1(BglIIBsrG1) (SEQ ID NO:54) containing a BglII restriction enzyme site. The DNA cassette Pdxs1upstream-Phps1-Pdxs1downstream was made by SOEing PCR among the Pdxs1 upstream homologous fragment, Phps1 promoter fragment and Pdxs1 downstream homologous fragment. The cassette Pdxs1upstream-Phps1-Pdxs1downstream was digested with XbaI and BglII, and cloned in Xba1/BglII sites of pGP704/sacBkan-trp to create pGP704/sacBkan-trp-Phps1-dxs1.

The chromosomal promoter replacement vector pGP704/sacBkan-trp-Pcat-ispFD was transferred into MWM1200 via triparental conjugation method as described in U.S. Pat. No. 7,232,666 Example 4, which is incorporated herein by reference. After conjugation, the chromosomal replacement of the ispFD genes' promoter with the Pcat promoter in MWM1200 was confirmed by PCR from the genomic DNA with the Pcat promoter specific primers ST-Pcat(ispFD) and SB-Pcat(ispFD), yielding the strain MWM1200+Pcat-ispFD. Then, the pGP704/sacBkan-trp-Phps1-dxs1 vector was transferred into MWM1200+Pcat-ispFD via triparental conjugation as before. After conjugation, the promoter replacement of the dxs1 gene promoter with the Phps1 promoter in MWM1200+Pcat-ispFD was confirmed by PCR from the genomic DNA with the Phps1 promoter specific primers ST-Phps1(dxs1) and SB-Phps1(dxs1), yielding DWS1044 (=MWM1200+Pcat-ispFD+Phps1-dxs1).

### Methylomonas Strain MWM1500

MWM1500 is a Methylomonas sp. 16a (ATCC PTA-2402) derivative with reduced glycogen synthase activity that was created by disrupting expression of the glgA gene in Methylomonas MWM1200. Methylomonas sp. MWM1500 has been deposited to ATCC under deposit number PTA-6888. Deletion of the glgA gene to produce the MWM1500 strain is described in U.S. Pat. No. 7,217,537 (Examples 3 and 4), which is incorporated herein by reference.

### Methylomonas Strain Growth and Culture Media

The standard conditions used for growth of Methylomonas sp. 16a (ATCC# PTA-2402) and derivatives thereof, as described in U.S. Pat. No. 6,689,601, which is incorporated herein by reference, are used in the following Examples for growth of Methylomonas sp. 16a, unless conditions are specifically described otherwise.

Methylomonas sp. 16a is typically grown in serum stoppered Wheaton bottles (Wheaton Scientific; Wheaton, Ill.) using a gas/liquid ratio of at least 8:1 (i.e., 20 mL of ammonium liquid “BTZ” growth medium in a Wheaton bottle of 160 mL total volume). The composition of the BTZ growth medium is described below. The standard gas phase for cultivation contains 25% methane in air, although methane concentrations can vary ranging from about 5-50% by volume of the culture headspace. These conditions comprise growth conditions and the cells are referred to as growing cells. In all cases, the cultures are grown at 30° C. with constant shaking in a rotary shaker (Thermo Scientific™ MaxQ 4000) unless otherwise specified.

### BTZ Medium for Methylomonas sp.

Methylomonas sp. 16a typically grows in a defined medium composed of only minimal salts; no organic additions such as yeast extract or vitamins are required to achieve growth. This defined medium known as BTZ medium (also referred to herein as “ammonium liquid medium”) consists of various salts mixed with Solution 1, as indicated in Tables 2 and 3. Alternatively, the ammonium chloride is replaced with 10 mM sodium nitrate to give “BTZ (nitrate) medium”, where specified. Solution 1 provides the composition for a 100-fold concentrated stock solution of trace minerals.

U.S. Pat. No. 6,689,601 describes growth of Methylomonas sp. 16a on defined medium with nitrate as the sole nitrogen source and up to 600 mM methanol, as the sole carbon source.

TABLE 2
Solution 1 composition

| | Molecular | Conc. | |
|---|---|---|---|
| | Weight | (mM) | g per L |
| Nitriloacetic acid | 191.10 | 66.90 | 12.80 |
| CuCl₂ × 2H₂O | 170.48 | 0.15 | 0.0254 |
| FeCl₂ × 4H₂O | 198.81 | 1.50 | 0.30 |
| MnCl₂ × 4H₂O | 197.91 | 0.50 | 0.10 |
| CoCl₂ × 6H₂O | 237.90 | 1.31 | 0.312 |
| ZnCl₂ | 136.29 | 0.73 | 0.10 |
| H₃BO₃ | 61.83 | 0.16 | 0.01 |
| Na₂MoO₄ × 2H₂O | 241.95 | 0.04 | 0.01 |
| NiCl₂ × 6H₂O | 237.70 | 0.77 | 0.184 |

*Mix the gram amounts designated above in 900 mL of H₂O, adjust to pH = 7.0, and add H₂O to a final volume of 1 L. Keep refrigerated.

TABLE 3
Ammonium Liquid Medium (BTZ)**

| | | Conc. | Amount | |
|---|---|---|---|---|
| | MW | (mM) | per L | |
| NH₄Cl | 53.49 | 10 | 0.537 | g |
| KH₂PO₄ | 136.09 | 3.67 | 0.5 | g |
| Na₂SO₄ | 142.04 | 3.52 | 0.5 | g |
| MgCl₂ × 6H₂O | 203.3 | 0.98 | 0.2 | g |
| CaCl₂ × 2H₂O | 147.02 | 0.68 | 0.1 | g |
| 1M HEPES (pH 7.0) | 238.3 | | 50 | mL |
| Solution 1 | | | 10 | mL |

**Dissolve in 900 mL H₂O. Adjust to pH = 7.0, and add H₂O to give a final volume of 1 L. For agar plates: Add 15 g of agarose in 1 L of medium, autoclave, cool liquid solution to 50° C., mix, and pour plates.

### Example 1

### Construction of an Isoprene Synthase (IspS) and Isopentenyl Diphosphate Isomerase (IDI) Expression Vector for Methylomonas

To create an ispS expression vector for Methylomonas, the chloramphenicol resistance gene promoter of the pBHR1 vector (MoBioTec, GmbH Göttingen, Germany) was used to direct expression of a modified P. alba ispS coding region. To construct the ispS expression vector, the GeneArt Seamless Cloning kit (Invitrogen™, Life Technologies, Grand Island, N.Y.) was used according to the manufacturer's protocol. The pBHR1 plasmid was amplified by PCR with Q5 High Fidelity DNA Polymerase, (Catalog #M0491, New England Biolabs Inc., Ipswich, Ma) according to the manufacturer's instructions to create a backbone vector. Primers for amplification were: BHR1 vec For (SEQ ID NO:55) and BHR1 vec Rev (SEQ ID NO:56). The PCR product was electrophoresed on a 1% agarose gel to confirm the molecular weight of the backbone vector, which was approximately 4600 bp.

The E. coli codon optimized sequence encoding a P. alba IspS polypeptide variant was used for expression (SEQ ID NO:3). The polypeptide variant has a 5′ truncation that is disclosed in U.S. Pat. No. 8,507,235, which is incorporated herein by reference, called a MEA variant. In addition the P. alba IspS variant has a substitution of cysteine for serine at position 288 (S288C) that is disclosed in WO2013063528, which is incorporated herein by reference. The variant protein is Seq ID NO:2. A DNA fragment (SEQ ID NO:57) containing the variant IspS encoding sequence (SEQ ID NO:3) flanked at the 5′ end by an EcoRI site and a putative RBS (GGAG) upstream of the ATG start and at the 3′ end by PmeI and XbaI restriction sites following the TAA stop codon was cloned into the backbone vector. The fragment was cloned into the pBHR1 backbone replacing the chloramphenicol resistance coding sequence.

Cloning reactions were transformed into chemically competent E. coli Top10 cells (Invitrogen™, Life Technologies, Grand Island, N.Y.) following the manufacturer's instructions. After outgrowth in SOC for 1 hour at 30° C. with shaking at 220 rpm, aliquots of the transformation mix were plated onto Luria Broth (LB) plates with 50 mg/L of kanamycin. Plates were incubated overnight at 30° C.

Transformants were screened by colony PCR to confirm the presence of the ispS coding region in the pBHR1 vector. A HotStar Master Mix Kit (Qiagen Inc, Valencia, Ca) was used for PCR amplification according to the manufacturer's protocol. PCR primers Cm prom seq for (SEQ ID NO:58) and IspS seq R2 (SEQ ID NO:59) yield an expected 782 bp PCR product. Colonies that amplified the correct sized PCR product were grown overnight in LB (Luria Broth) with 50 mg/L kanamycin. Plasmid DNA from the transformants was prepared using a QIAprep Spin Miniprep Kit (Qiagen Inc., Valencia, Calif.) according to manufacturer's protocols. Plasmid DNA was sequenced to confirm the sequence of the resulting plasmid pBHR1-ispS_S288C with primers Cm prom seq for (SEQ ID NO:58), IspS seq F1 (SEQ ID NO:60), IspS seq F2 (SEQ ID NO:61), IspS seq F3 (SEQ ID NO:62), IspS seq R1 (SEQ ID NO:63), IspS seq R2 (SEQ ID NO:59), IspS seq R3 (SEQ ID NO:64), and pBHR1 do Cm rev (SEQ ID NO:65).

A Methylomonas expression vector was constructed containing coding regions for isoprene synthase, as described above, and the S. cerevisiae coding region for isopentenyl diphosphate isomerase (IDI; SEQ ID NO:7), both under control of the chloramphenicol resistance gene promoter. A DNA fragment (SEQ ID NO:66) having at the 5′ end of the idi coding region a PmeI restriction site followed by a putative RBS (GGAG) upstream of the ATG start, and at the 3′ end of the coding region after the TAA stop codon, an XbaI restriction site was cloned into the pBHR1-ispS vector. The pBHR1-ispS vector and the DNA fragment were digested with PmeI and XbaI (New England Biolabs, Inc). Both the vector and the idi coding region fragment were purified using a DNA Clean & Concentrator™ kit (Zymo Research Corporation, Irvine, Calif.) per manufacturer's instructions. The vector and insert were ligated with T4 DNA ligase (New England Biolabs, Inc) according to manufacturer's protocol at 16 C overnight. The ligation was transformed into chemically competent E. coli Top10 cells (Invitrogen™, Life Technologies, Grand Island, N.Y.) following manufacturer's instructions. After outgrowth in SOC for 1 hour at 30° C. with shaking at 220 rpm, aliquots of the transformation mix were plated onto Luria Broth (LB) plates with 50 mg/L of kanamycin. Plates were incubated overnight at 30° C.

To confirm the presence of the idi coding region in the vector, transformants were screened by PCR amplification with primers ID seq F1 (SEQ ID NO:67) and pBHR1 dn Cm rev (SEQ ID NO:65). Colonies that amplified the expected 736 bp PCR product were grown overnight in LB with 50 mg/L kanamycin. Plasmid DNA from transformants was prepared with QIAprep Spin Miniprep Kit (Qiagen Inc., Valencia, Calif.) according to the manufacturer's protocols. Plasmid DNA was sequenced to confirm the sequence of the resulting pBHR1-ispS_S288C-idi vector with primers ispS seq F3 (SEQ ID NO:62), ID seq F1 (SEQ ID NO:67), ID seq R1 (SEQ ID NO:68) and and pBHR1 dn Cm rev (SEQ ID NO:65).

### Example 2

### Prophetic

### Introduction of IspS and IDI Expression Vector into Methylomonas by Triparental Mating

The mobilization of pBHR1-ispS_S288C, pBHR1-ispS_S288C-idi, and pBHR1 (as control), vector DNAs into Methylomonas is through conjugation in tri-parental matings. The conjugative plasmid pRK2013 (ATCC No. 37159), which resides in a strain of E. coli, facilitates the DNA transfer as a helper plasmid.

### Growth of Methylomonas sp on Methane

Growth of strains Methylomonas sp. 16a, MWM1900, DWS1044, and MWM1500 (see General Methods) is initiated by inoculation of −80° C. frozen stock cultures into 20 mL of BTZ medium containing 25% methane (General Methods). The cultures are grown at 30° C. with aeration until the density of the culture is saturated. Each saturated culture is used to inoculate 100 mL of fresh BTZ medium containing 25% methane. The 100 mL culture is grown at 30° C. with aeration until the culture reaches an OD₆₀₀ between 0.7 to 0.8. To prepare the cells for the tri-parental mating, the cells are washed twice in an equal volume of BTZ medium. The cell pellets are re-suspended in the minimal volume needed (approximately 200 to 250 μL). Approximately 40 μL of the re-suspended cells is used in each tri-parental mating experiment.

### Growth of Methylomonas sp. on Methanol

Growth of Methylomonas sp. 16a (ATCC PTA-2402), MWM1900, MWM1500, and DWS1044 is initiated by inoculation of −80° C. frozen stock cultures into 20 mL of BTZ nitrate medium containing 100 mM methanol (General Methods). The cultures are grown at 30° C. with aeration until the density of the cultures is around an OD₆₀₀ of 1.0. These cultures are used to inoculate 50 mL of fresh BTZ nitrate medium containing 80 mM methanol. The 100 mL cultures are grown at 30° C. with aeration until the cultures reach an OD₆₀₀ between 0.7 to 0.8. To prepare the cells for the tri-parental mating, the cells are washed twice in an equal volume of BTZ medium. The cell pellets are re-suspended in the minimal volume needed (approximately 200 to 250 μL). Approximately 40 μL of the re-suspended cells are used in each tri-parental mating experiment.

Growth of the Escherichia coli Donor and Helper Cells

Isolated colonies of E. coli donor strains carrying pBHR1, pBHR1-ispS_S288C, or pBHR1-ispS_S288C-idi (donor cells), and the E. coli helper strain containing the conjugative plasmid pRK2013 (helper cells), are used to separately inoculate 5 mL of LB broth containing 25 μg/mL Kan. These cultures are grown overnight at 30° C. with aeration. The following day, the E. coli donor cells, carrying pBHR1, pBHR1-ispS_S288C, or pBHR1-ispS_S288C-idi, and E. coli helper cells are mixed together and incubated at 30° C. for about 2 hours. Subsequently, the cells are washed twice in equal volumes of fresh LB broth to remove the antibiotics.

Tri-Parental Mating: Mobilization of the Donor Plasmid into Methylomonas Strains

### Methane Procedure: Approximately 40 μL of the Re-Suspended

Methylomonas cells are used to re-suspend the combined E. coli donor and helper cell pellets after the washing. After thoroughly mixing the cells, the cell suspension is spotted onto BTZ agar plates containing 0.05% yeast extract. The plates are incubated at 30° C. for 3 days in a jar containing 25% methane.

Following the third day of incubation, the cells are scraped from the plate and re-suspended in BTZ broth. The entire cell suspension is plated onto several BTZ agar plates containing Kan at 50 mg/L (Kan⁵⁰). The plates are incubated at 30° C. in a jar containing 25% methane until colonies are visible (approximately 4-7 days).

Individual colonies are streaked onto fresh BTZ+Kan⁵⁰ agar plates and incubated for 1-2 days at 30° C. in the presence of 25% methane. These cells are used to inoculate bottles containing 20 mL of BTZ and 25% methane. After overnight growth, 5 mL of the culture is concentrated by centrifugation using a tabletop centrifuge. Then, to rid the cultures of E. coli cells that are introduced during the tri-parental mating, the cells are inoculated into 20 mL of BTZ liquid medium containing nitrate (10 mM) as the nitrogen source, methanol (200 mM), and 25% methane, and grown overnight at 30° C. with aeration. Cells from these BTZ (nitrate) cultures are again inoculated into BTZ and 25% methane, and grown overnight at 30° C. with aeration. The cultures are monitored for E. coli growth by plating onto LB agar plates to verify the success of the E. coli elimination.

### Methanol Procedure: Approximately 40 μL of the Re-Suspended

Methylomonas cells are used to re-suspend the combined E. coli donor and helper cell pellets. After thoroughly mixing the cells, the cell suspension is spotted onto BTZ agar plates containing 0.05% yeast extract and 80 mM methanol. The plates are incubated at 30° C. for 3 days in a jar.

Following the third day of incubation, the cells are scraped from the plate and re-suspended in BTZ nitrate broth. The entire cell suspension is plated onto several BTZ nitrate agar plates containing Kan⁵⁰ and 80 mM methanol. The plates are incubated at 30° C. in a jar until colonies are visible (approximately 7-12 days).

Individual colonies are streaked onto fresh BTZ nitrate+Kan⁵⁰ 80 mM methanol agar plates and incubated 2-3 days at 30° C. These cells are used to inoculate bottles containing 20 mL of BTZ nitrate containing 80 mM methanol. After overnight growth, 5 mL of the culture is concentrated by centrifugation using a tabletop centrifuge. Then, to rid the cultures of E. coli cells that are introduced during the tri-parental mating, the cells are inoculated into 20 mL of BTZ nitrate liquid medium containing methanol (200 mM) and grown overnight at 30° C. with aeration. Cells from the BTZ (nitrate) cultures are again inoculated into BTZ nitrate and 80 mM methanol and grown overnight at 30° C. with aeration. The cultures are monitored for E. coli growth by plating onto LB agar plates, to verify the success of the E. coli elimination.

### Example 3

### Prophetic

### Production of Isoprene from Methane or Methanol by Methylomonas Expressing IspS and IDI

Cultures of Methylomonas cells carrying either pBHR1 or pBHR1-ispS-idi_S288C are grown according to the conditions described in General Methods. Serum bottles are incubated at 30° C. and regularly sampled for headspace analysis. Cell growth is monitored by OD₆₀₀. The headspace is sampled by solid phase microextraction (SPME). The SPME fiber is exposed to headspace in the bottle for 10 minutes, then injected onto a 30 m×0.25 um HP-5MS column. The detector is set to scan mode from m/z 29 to 250. Software is used to extract for m/z 67 ion, characteristic of isoprene. Isoprene elutes at 1.63 minutes under the conditions run. An authenticated standard is used to confirm the spectrum and retention time.

Increased isoprene is observed in the headspace of Methylomonas cells carrying pBHR1-ispS_S288C-idi as compared the headspace of Methylomonas cells carrying the empty vector pBHR1, and in comparison to Methylomonas cells with no introduced plasmid. Isoprene production is greater by pBHR1-ispS_S288C-idi transformants of MWM1900 (having disruption in C₃₀ carotenoid production), MWM1500 (having disruption in C₃₀ carotenoid production and in glycogen synthase), and DWS1044 (having disruption in C₃₀ carotenoid production and increase in DXP pathway gene expression) than by transformants of the original Methylomonas sp. 16a.

### Example 4

### Construction of Isoprene Synthase (IspS) and Isopentenyl Diphosphate Isomerase (IDI) Expression Vectors for Methylomonas

The coding regions for ispS and IDI were cloned into vector pBHR1 (MoBioTec, GmbH Göttingen, Germany) under the control of three different promoter constructs: (i) the chloramphenicol resistance gene promoter (P_{cat}) from the pBHR1 vector which has the same sequence as SEQ ID NO:5, (ii) the native hps (hexulose phosphate synthase) gene promoter (Phps1 promoter) from Methylomonas sp. 16a, (SEQ ID NO:49), and (iii) a dual promoter constructed by fusing the P_{cat} promoter and a Pₕₚₛ_{_{—}}_{NcoI} promoter (SEQ ID NO:69) to create the Pcat_Phps promoter (SEQ ID NO:70).

The P. alba IspS variant MEA S288C codon-optimized for E. coli expression (SEQ ID NO:3) was first cloned under the control of each of the 3 promoters creating expression plasmids: pBHR1-P_{cat}-ispS_S288C (SEQ ID NO:71), pBHR1-Pₕₚₛ-ispS_S288C (SEQ ID NO:72), and pBHR1-P_{cat}Pₕₚₛ-iSp_S_S288C (SEQ ID NO:73). The IspS coding region in all three vectors had an ATG start codon as part of an NcoI site that is downstream of a putative RBS (GGAG). The 3′ end of the ispS coding sequence fragment contained a TAA stop codon followed by PmeI and XbaI restriction sites. Next, the yeast idi coding region (SEQ ID NO:7) was cloned downstream of the ispS S288C gene in each of the three expression vectors. A DNA fragment (SEQ ID NO:66) having at the 5′ end of the idi coding region a PmeI restriction site followed by a putative RBS (GGAG) upstream of the ATG start, and at the 3′ end of the coding region after the TAA stop codon, an XbaI restriction site was cloned into the ispS expression plasmids above as a PmeI-XbaI fragment. The resulting plasmids were named: pBHR1-P_{cat}-ispS_S288C-idi, pBHR1-Pₕₚₛ-ispS_S288C-idi, and pBHR1-P_{cat} Pₕₚₛ-ispS_S288C-idi.

An ispS_S288C-idi cassette was codon-optimized for Methylomonas and cloned into vector pBHR1 under the control of the dual promoter P_{cat}Pₕₚₛ. The Methylomonas codon optimized ispS_S288C coding region (SEQ ID NO:4) had an ATG start codon as part of an NcoI site that is downstream of a putative RBS (GGAG). The 3′ end of the ispS sequence contained a TAA stop codon followed by a PmeI site. The Methylomonas codon optimized idi coding region (SEQ ID NO:8) was downstream of the ispS_S288C gene. The idi gene fragment consisted of a PmeI restriction site followed by a putative RBS (GGAG) upstream of the ATG start. At the 3′ end of the idi coding region sequence, downstream of the TAA stop codon, was an XbaI restriction site. The resulting plasmid was named pBHR1-P_{cat}Pₕₚₛ-ispS_S288C-idi_{—}2.0 (SEQ ID NO:74).

An empty vector control was also constructed from pBHR1. Vector pBHR1 was digested with DraI and ScaI removing about 500 bp of the cat coding region. The 4800 bp digested vector was gel purified and religated to create pDCm1.

### Example 5

### Introduction of IspS and 101 Expression Vectors into Methylomonas by Triparental Mating

The mobilization of pBHR1-P_{cat}-ispS_S288C-idi, pBHR1-Pₕₚₛ-ispS_S288C-idi, pBHR1-Pᵣₐₜ Pₕₚₛ-ispS_S288C-idi, pBHR1-P_{cat} Pₕₚₛ-ispS_S288C-idi 2.0 and the control, pDCm1 into Methylomonas was through conjugation in tri-parental matings. The conjugative plasmid pRK2073 (described in U.S. Pat. No. 7,504,236) which resides in a strain of E. coli, facilitated the DNA transfer as a helper plasmid.

### Growth of Methylomonas sp. on Methanol

Growth of Methylomonas sp. 16a (ATCC PTA-2402) for conjugation was initiated by inoculating several colonies from a streaked BTZ nitrate plate into 20 ml BTZ nitrate medium containing 50 mM methanol in a 160 ml serum bottle. The culture was grown at 30° C. with aeration overnight. The overnight culture was then used to inoculate 50 ml of fresh BTZ nitrate medium containing 50 mM methanol in a 500 ml serum bottle to a starting OD₆₀₀=0.01. The 50 ml cultures were grown overnight at 30° C. with aeration until they reached an OD₆₀₀ of about 0.7. The cells were then harvested by centrifugation at 4000 rpm for 15 min. Cell pellets were washed twice in an equal volume of BTZ medium and centrifuged as before. After the last wash, a cell pellet from 50 ml of culture was re-suspended in approximately 100 μl BTZ nitrate medium.

Growth of the Escherichia coli Donor and Helper Cells

The E. coli donor strains carrying pDCm1, pBHR1-P_{cat}-ispS_S288C-idi, pBHR1-Pₕₚₛ-ispS_S288C-idi, pBHR1-P_{cat}Pₕₚₛ-ispS_S288C-idi, and pBHR1-P_{cat}Pₕₚₛ-ispS_S288C-idi_{—}2.0 were grown overnight in 25 ml LB containing 50 mg/l kanamycin. The E. coli helper strain containing the helper plasmid pRK2073 was grown overnight in 25 ml LB broth containing 25 mg/l chloramphenicol. After overnight growth, the E. coli donor cells and helper cells were each sub-cultured into 30 ml LB containing the appropriate antibiotic to an OD₆₀₀ of approximately 0.2 and grown at 30° C. with shaking to an OD₆₀₀=0.7-1.2. For each conjugation, 3 ml of the donor strain and 1.5 ml of the helper strain were centrifuged at 4000 rpm for 5 min and the pellets washed once in LB to remove the antibiotics. The cells were then centrifuged and again re-suspended in 3 ml LB for the donor strain and 1.5 ml for the helper strain. The donor and helper cells were then mixed together and incubated at 30° C. while the Methylomonas cells were harvested. Once the Methylomonas culture was harvested and re-suspended, the mixed E. coli cells were then pelleted by centrifugation.

Tri-Parental Mating: Mobilization of the Donor Plasmid into Methylomonas Strains

### Methanol Procedure:

Approximately 80 μl of the re-suspended Methylomonas cells were mixed with the E. coli donor and helper cell pellet. After gentle mixing, the cell suspension was spotted onto BTZ nitrate agar plates containing 0.05% yeast extract. The plates were air-dried and then inverted. The lid of each plate received 20 μl of methanol (approximately 20 mM). The plates were incubated at 30° C. for 3 days in an AnaeroPack System 2.5 L rectangular jar (Mitsubishi Gas Chemical Co., Inc., Tokoyo, Japan). Following the third day of incubation, the cells were scraped from the plates and re-suspended in 600 μl BTZ nitrate broth. 100 μl neat and 100 μl of a 10⁻¹ dilution of the suspension were plated onto several BTZ nitrate agar plates containing 50 mg/l kanamycin. Plates were inverted and 20 μl of methanol was added to each lid. The plates were incubated at 30° C. in a jar until colonies were visible (approximately 5-12 days). More methanol was added to the lid about every 3 days. Individual colonies were patched onto fresh BTZ nitrate agar plates containing 50 mg/l kanamycin with 20 μl methanol in the lid and incubated 3-4 days at 30° C. Next, the patches were streaked for single colonies on fresh BTZ nitrate+50 mg/l kanamycin agar plates with 20 μl methanol in the lid and incubated 7-12 days at 30° C. Then, single colonies were patched onto fresh BTZ nitrate+50 mg/l kanamycin agar plates with 20 μl methanol in the lid. Cells from these patches were patched onto LB plates with no antibiotics and incubated for 2 days at 37° C. to check for E. coli contamination. Patches that were free of E. coli were streaked onto fresh BTZ nitrate+50 mg/l kanamycin agar plates with 20 μl methanol in the lid and incubated 3-5 days at 30° C. Finally, individual colonies were patched onto BTZ nitrate+50 mg/l kanamycin plates and again onto LB plates to verify the absence of E. coli contamination.

### Example 6

### Production of Isoprene from Methanol by Methylomonas Expressing IspS and Idi

To determine whether heterologous expression of IspS and Idi enabled Methylomonas to convert methanol to isoprene, GC/MS was used to monitor isoprene levels in the headspace of Methylomonas cultures grown on methanol. Isoprene production by Methylomonas sp. 16a cells harboring plasmids pBHR1-P_{cat}-ispS_S288C-idi, pBHR1-Pₕₚₛ-ispS_S288C-idi, pBHR1-P_{cat}Pₕₚₛ-iSPS_S288C-idi, pBHR1-P_{cat}Pₕₚₛ-ispS_S288-idi_{—}2.0 or pDCm1 (as control) was compared after growth on methanol as the carbon source. Growth of these strains was initiated by inoculating patches from BTZ (nitrate) plates into 20 ml BTZ (nitrate) liquid medium containing 50 mg/l kanamycin and 80 mM methanol in stoppered 160 ml serum bottles. After incubation for 24 h at 30° C. with shaking at 140 rpm, cells were sub-cultured to an OD₆₀₀ of 0.02 into 20 ml BTZ (nitrate) medium containing 50 mg/l kanamycin and 80 mM methanol in stoppered 160 ml serum bottles. Each strain was sub-cultured in triplicate. Cultures were incubated at 30° C. with shaking at 180 rpm. After 24 h, another 80 mM methanol was injected into each bottle and incubation continued. After a total of 43 h incubation, 1 ml was removed from the headspace of each serum bottle using a gas-tight syringe and injected into an Agilent 7890A GC fitted with a 30 m×0.25 mm×1 um HP5-MS GC column. The GC inlet was held at 250° C. at a 10:1 split. Helium was the carrier gas at a flow rate of 2 mL/min. The GC method was run in isothermal mode at 70° C. Detection was accomplished with a 5973 MSD unit. Software was used to extract for m/z 67 ion, characteristic of isoprene. Isoprene elutes at 1.63 minutes under the conditions run (as confirmed using an isoprene standard). The height of the peak at 1.63 min was recorded for each sample, and normalized to the OD₆₀₀ of the culture (Table 4).

TABLE 4
Isoprene production by Methylomonas strains expressing IspS
and Idi after 43 h growth on methanol

| | Peak height/ | Fold change over |
|---|---|---|
| Plasmid | OD₆₀₀^{a} | pDCm1 control^{b} |
| pDCm1 (control) | 368 ± 14 | n/a |
| pBHR1-P_{cat}-ispS_S288C-idi | 456 ± 38 | 1.2 |
| pBHR1-Pₕₚₛ-ispS_S288C-idi | 577 ± 52 | 1.6 |
| pBHR1-P_{cat} Pₕₚₛ-ispS_S288C-idi | 825 ± 69 | 2.2 |
| pBHR1-P_{cat} Pₕₚₛ-ispS_S288C-idi_2.0 | 560 ± 12 | 1.5 |

^{a}GC peak height at 1.63 min retention time, normalized to OD₆₀₀ of culture. Data are averages from triplicate experiments, shown ± standard deviation.
^{b}Calculated by dividing (peak height/OD₆₀₀) value for test culture by (peak height/OD₆₀₀) value for pDCM1 control

Headspace samples from Methylomonas cultures carrying pBHR1-Pₕₚₛ-ispS_S288C-idi, pBHR1-P_{cat}-ispS_S288C-idi, pBHR1-P_{cat}Pₕₚₛ-ispS_S288C-idi, or pBHR1P_{cat}Pₕₚₛ-ispS_S288C-idi_{—}2.0 all produced higher GC peaks corresponding to isoprene than did samples from the control culture harboring the empty vector pDCm1 (Table 4). Isoprene production was greatest for cells containing pBHR1-P_{cat}Pₕₚₛ-ispS_S288C-idi (expressing ispS and idi from the P_{cat}Pₕₚₛ dual promoter). These data indicate that expression of IspS and Idi in Methylomonas enables increased isoprene production by Methylomonas grown on methanol as the sole carbon source. [Note that some isoprene is produced even in the strain not expressing IspS and Idi. Many bacteria produce low levels of isoprene, despite lacking obvious homologs of IspS].

### Example 7

### Production of Isoprene from Methane by Methylomonas Expressing IspS and Idi

To determine whether heterologous expression of ispS and idi enabled Methylomonas to convert methane to isoprene, GC was used to compare isoprene production by Methylomonas sp. 16a cells harboring plasmids pBHR1-P_{cat}-ispS_S288C-idi, pBHR1-Pₕₚₛ-ispS_S288C-idi, pBHR1-P_{cat}PₕₚₛS_S288C-idi pBHR1P_{cat}PₕₚₛS_S288C-idi_{—}2.0 or pDCm1 (as control) after growth on methane as the carbon source. Growth of these strains was initiated by inoculating patches from BTZ (nitrate) plates into 20 ml BTZ (nitrate) liquid medium containing 50 mg/I kanamycin and 80 mM methanol in stoppered 160 ml serum bottles. After incubation for 24 h at 30° C. with shaking at 180 rpm, cells were subcultured to an OD₆₀₀ of 0.02 into 20 ml BTZ (NH₄) medium containing 50 mg/l kanamycin in stoppered 160 ml serum bottles. Each strain was subcultured in triplicate. Serum bottles were evacuated, purged with nitrogen, evacuated again, and then filled to 5 psig with a mixture of 50% CH₄/21% O₂/29% N₂ and incubated at 30° C. with shaking at 180 rpm. After 48 h, 1 ml was removed from the headspace of each serum bottle using a gas-tight syringe and injected into an Agilent 7890A GC fitted with a 30 m×0.25 mm×1 um HP5-MS GC column. The GC inlet was held at 250° C. at a 10:1 split. Helium was the carrier gas at a flow rate of 2 mL/min. The GC method was run in isothermal mode at 70° C. Detection was accomplished with a 5973 MSD unit. Software was used to extract for m/z 67 ion, characteristic of isoprene. Isoprene elutes at 1.63 minutes under the conditions run (as confirmed using an isoprene standard). The height of the peak at 1.63 min was recorded for each sample, and normalized to the OD₆₀₀ of the culture (Table 5).

TABLE 5
Isoprene production by Methylomonas strains expressing IspS and
Idi after 48 h growth on methane

| | Peak height/ | Fold change over |
|---|---|---|
| Plasmid | OD₆₀₀^{a} | pDCm1 control^{b} |
| pDCm1 (control) | 504 ± 122 | n/a |
| pBHR1-P_{cat}-ispS_S288C-idi | 596 ± 99 | 1.2 |
| pBHR1-Pₕₚₛ-ispS_S288C-idi | 706 ± 175 | 1.4 |
| pBHR1-P_{cat} Pₕₚₛ-ispS_S288C-idi | 1307 ± 313 | 2.6 |
| pBHR1-P_{cat} Pₕₚₛ-ispS_S288C-idi_2.0 | 961 ± 31 | 1.9 |

^{a}GC peak height at 1.63 min retention time, normalized to OD₆₀₀ of culture. Data are averages from triplicate experiments, shown ± standard deviation.
^{b}Calculated by dividing (peak height/OD₆₀₀) value for test culture by (peak height/OD₆₀₀) value for pDCM1 control.

Headspace samples from Methylomonas cultures carrying pBHR1-Pₕₚₛ-ispS_S288C-idi, pBHR1-P_{cat}Pₕₚₛ-ispS_S288C-idi, or pBHR1-P_{cat}Pₕₚₛ-ispS_S288C-idi_DNA2.0 all produced higher GC/MS peaks corresponding to isoprene than did samples from the control culture harboring the empty vector pDCm1 (Table 5). Isoprene production on methane was greatest for cells containing pBHR1-P_{cat}Pₕₚₛ-ispS_S288C-idi (expressing ispS and idi from the P_{cat}Pₕₚₛ dual promoter). These data indicate that expression of IspS and Idi in Methylomonas enables increased isoprene production by Methylomonas grown on methane as the sole carbon source. The fold-increase in isoprene production relative to the pDCm1 control was broadly similar for cells grown on methane (Table 5) and methanol (Table 4).

The effect of replenishing the methane gas mixture in the culture headspace on isoprene production by Methylomonas was also measured. Methylomonas cells harboring either pBHR1-P_{cat}Pₕₚₛ-ispS_S288C-idi or the control plasmid pDCm1 were inoculated into 20 ml BTZ (nitrate) liquid medium containing 50 mg/l kanamycin and 50 mM methanol in stoppered 160 ml serum bottles, incubated for 24 h at 30° C., then subcultured to an OD₆₀₀ of 0.01 into 20 ml BTZ medium containing 50 mg/l kanamycin in stoppered 160 ml serum bottles. Serum bottles were purged with nitrogen then filled to 5 psig with a mixture of 50% CH₄/21% O₂/29% N₂. One set of bottles was incubated for 48 h at 30° C. with shaking at 180 rpm. 1 ml headspace was then removed and analyzed by GC as described above, and then the bottles were purged with nitrogen and re-gassed with 50% CH₄/21% O₂/29% N₂ to 5 psig. The bottles were incubated for a further 24 h (72 h total incubation), then another 1 ml headspace was removed and analyzed by GC/MS. A second set of bottles was incubated for 72 h at 30° C. with shaking at 180 rpm (without sampling or re-gassing), then 1 ml headspace was removed and analyzed by GC/MS. Data shown in Table 6 show that after 72 h, isoprene production (as measured by the fold-change in peak height/OD₆₀₀ vs the pDCm1 control) was greater for cultures that had been re-gassed after 48 h than for the same cultures at 48 h before re-gassing, or for cultures that were incubated for 72 h with no re-gassing. Incubation for 72 h also resulted in production of slightly more isoprene than did incubation for 48 h, even without re-gassing. These data indicate that production of isoprene from methane can be increased by incubating cultures for longer periods of time (72 h as opposed to 48 h), and by re-gassing cultures during incubation (e.g. after 48 h).

TABLE 6
Effect of re-gassing cultures on isoprene production by Methylomonas expression IspS and Idi

| | | 48 h (before re-gassing) | | | | 72 h (24 h after regassing) | | | |
|---|---|---|---|---|---|---|---|---|---|
| | Re- | | | | Fold change | | | | Fold change |
| | gassed | | | Peak | over | | | Peak | over |
| | after | Peak | | height/ | pDCm1 | Peak | | height/ | pDCm1 |
| Plasmid | 48 h? | height | OD₆₀₀ | OD₆₀₀ | control | height | OD₆₀₀ | OD₆₀₀ | control^{b} |
| pDCm1 | No | | | | | 1100 | 1.504 | 731 | |
| (control) | | | | | | | | | |
| pBHR1-P_{cat} | No | | | | | 3100 | 1.368 | 2266 | 3.1 |
| Pₕₚₛ- | | | | | | | | | |
| ispS_S288C- | | | | | | | | | |
| idi | | | | | | | | | |
| pDCm1 | Yes | 820 | 1.024 | 801 | | 830 | 2.316 | 358 | |
| (control) | | | | | | | | | |
| pBHR1-P_{cat} | Yes | 2500 | 1.292 | 1935 | 2.42 | 4000 | 2.776 | 1441 | 4.02 |
| Pₕₚₛ- | | | | | | | | | |
| ispS_S288C- | | | | | | | | | |
| idi | | | | | | | | | |

### Example 8

### Quantification of Isoprene Production by Solid Phase Microextraction

To confirm that the product detected by GC/MS was isoprene, and to more precisely quantify isoprene in the culture headspace, solid phase microextraction (SPME) was used to sample the headspace of Methylomonas cultures carrying either the IspS and Idi expression plasmid pBHR1-P_{cat}Pₕₚₛ-ispS_S288C-idi or the control plasmid pDCm1 after growth on methane or methanol.

For growth on methane, cells were first inoculated into 20 ml BTZ (nitrate) liquid medium containing 50 mg/l kanamycin and 50 mM methanol in stoppered 160 ml serum bottles, incubated for 24 h at 30° C. with shaking at 180 rpm, and then were sub-cultured to an OD₆₀₀ of 0.01 into 20 ml BTZ medium containing 50 mg/l kanamycin in stoppered 160 ml serum bottles. Serum bottles were purged with nitrogen, then filled to 5 psig with a mixture of 50% CH₄/21% O₂/29% N₂ and incubated at 30° C. with shaking at 180 rpm.

For growth on methanol, cells were inoculated into 20 ml BTZ (nitrate) liquid medium containing 50 mg/l kanamycin and 80 mM methanol in stoppered 160 ml serum bottles, incubated for 24 h at 30° C. with shaking at 160 rpm, and then were subcultured to an OD₆₀₀ of 0.02 into 20 ml BTZ medium (nitrate) containing 50 mg/l kanamycin and 80 mM methanol in stoppered 160 ml serum bottles. Bottles were incubated as before. After approximately 24 hours of incubation, another 80 mM methanol was added to the stoppered bottles by syringe and incubation continued.

After incubation for 48 h, the headspace of each bottle was sampled by SPME using a 75 μm CAR/PDMS Fused Silica SPME fiber, which was conditioned by heating at 275° C. under helium flow for 15 min. The SPME fiber was inserted into the bottle through the septum and allowed to equilibrate with the headspace for 30 min, then removed from the bottle and injected onto a chromatographic column (RTX-1 60 m×0.320 mm×3 μm) in an Agilent 7890A/5975C GC/MS. The fiber was held in the inlet for 2 minutes at 250° C. Desorbed material from the fiber was collected onto the chromatographic column (RTX-1 60 m×0.320 mm×3 μm) by cooling the oven to −35° C. After the desorption period, the fiber was removed from the inlet and the oven was heated at 20° C. per minute to a final temperature of 250° C. Software was used to extract for m/z 67 ion, characteristic of isoprene. To determine production of isoprene in experimental samples, peak retention times and mass spectra of the samples were compared to an isoprene standard. Isoprene elutes at 10.2 minutes under the conditions run. A peak at 10.2 minutes corresponding to isoprene was detected from headspace samples from all cultures (those harboring pBHR1-P_{cat}Pₕₚₛ-ispS_S288C-idi or the control plasmid pDCm1) after growth on methane or methanol. Quantification of the peaks showed that 2.7 to 2.8-fold more isoprene was produced by cells harboring pBHR1-P_{cat}Pₕₚₛ-ispS_S288C-idi than by those containing the control plasmid pDCm1 (Table 7). This fold-increase in isoprene production is consistent with that observed by GC without SPME (Tables 4, 5 and 6). These data confirm that isoprene is produced by Methylomonas expressing IspS and Idi when grown on methane or methanol.

TABLE 7
Quantification of isoprene production by Methylomonas
expressing IspS and Idi after growth on methane or methanol

| Carbon | | Mass isoprene per | Fold change over |
|---|---|---|---|
| Source | Plasmid | volume broth (ng/ml) | pDCm1 control |
| Methane | pDCm1 (control) | 20.3 | |
| | pBHR1-P_{cat} Pₕₚₛ- | 56.4 | 2.77 |
| | ispS_S288C-idi | | |
| Methanol | pDCm1 (control) | 19.5 | |
| | pBHR1-P_{cat} Pₕₚₛ- | 53.3 | 2.73 |
| | ispS_S288C-idi | | |

What is claimed is:

1. Recombinant methylotrophic cells comprising:
a) at least one heterologous nucleic acid molecule encoding an isoprene synthase polypeptide; and
b) at least one genetic modification which increases isopentenyl diphosphate isomerase activity in the cells as compared with isopentenyl diphosphate isomerase activity in the cells lacking said genetic modification:
wherein the cells produce more isoprene when grown in culture conditions comprising at least one of methane and methanol as a carbon source, as compared to the cells without (a) and (b).

2. The recombinant cells of claim 1 wherein the methylotrophic cells are methanotrophic bacterial cells.

3. The recombinant cells of claim 1 wherein the methylotrophic cells are methylotrophic yeasts.

4. The methylotrophic yeasts of claim 3 selected from the group of genera consisting of Candida, Hansenula, Pichia, Torulopsis, and Rhodotorula.

5. The cells of claim 1 wherein the at least one genetic modification of (b) is accomplished by a process selected from the group consisting of:
a) increasing expression of an endogenous polypeptide having isopentenyl diphosphate isomerase activity;
b) expressing a heterologous nucleic acid molecule encoding a polypeptide having isopentenyl diphosphate isomerase activity; and
c) both (a) and (b).

6. The cells of claim 1 wherein the isoprene synthase polypeptide belongs to the enzyme classification group EC 4.2.3.27.

7. The cells of claim 1 wherein the isopentenyl diphosphate isomerase activity is provided by an isopentenyl diphosphate isomerase polypeptide belonging to the enzyme classification group EC 5.3.3.2.

8. The cells of claim 2, wherein the methanotrophic bacterial cells belong to a genera selected from the group consisting of Methylomonas, Methylobacter, Methylococcus, Methylosinus, Methylocyctis, and Methylomicrobium.

9. The cells of claim 1, further comprising at least one genetic modification which increases activity of at least one enzyme of the DXP pathway, other than isopentenyl diphosphate isomerase activity.

10. The cells of claim 9, wherein the enzyme of the DXP pathway is selected from 1-deoxyxylulose-5-phosphate synthase, 1-deoxy-D-xylulose-5-phosphate reductoisomerase, 2C-methyl-D-erythritol cytidyltransferase, 4-diphosphocytidyl-2-C-methyl-D-erythritol kinase, 2C-methyl-D-erythritol 2,4-cyclodiphosphate synthase, 1-hydroxyl-2-methyl-2-(E)-butenyl-4-diphosphate synthase, 4-hydroxy-3-methylbut-2-enyl diphosphate reductase, and combinations thereof.

11. The cells of claim 1 or 9, further comprising at least one genetic modification which decreases activity of at least one endogenous gene encoding an enzyme in a pathway downstream of IPP and DMAPP.

12. The cells of claim 11, wherein the pathway downstream of IPP and DMAPP is a pigment biosynthesis pathway.

13. The cells of claim 11, wherein the endogenous gene encoding an enzyme in a pathway downstream of IPP and DMAPP is selected from ispA, phs, crtM, ald, crtN1, crtN2, and crtN3.

14. The cells of claim 1 or 9, further comprising at least one genetic modification which increases activity of at least one enzyme in a pathway for at least one of methane assimilation and methanol assimilation.

15. A method for constructing recombinant methylotrophic cells that produce isoprene comprising:
a) introducing at least one heterologous nucleic acid molecule encoding an isoprene synthase polypeptide; and
b) making at least one genetic modification which increases isopentenyl diphosphate isomerase activity in the cells as compared with isopentenyl diphosphate isomerase activity in the cells lacking said genetic modification.

16. The method of claim 15 further comprising at least one of:
a) making at least one genetic modification which increases activity of at least one enzyme of the DXP pathway, other than isopentenyl diphosphate isomerase activity, wherein the activity is higher than in cells without the modification;
b) making at least one genetic modification which decreases activity of at least one endogenous gene encoding an enzyme in a pathway downstream of IPP and DMAPP; and
c) making at least one genetic modification which increases activity of at least one enzyme in a pathway for at least one of methane assimilation and methanol assimilation.

17. A method for producing isoprene comprising:
a) providing recombinant methylotrophic cells comprising:
i) at least one heterologous nucleic acid molecule encoding an isoprene synthase polypeptide; and
ii) at least one genetic modification which increases isopentenyl diphosphate isomerase activity in the cells as compared with isopentenyl diphosphate isomerase activity in the cell lacking said genetic modification, and
b) growing the cells of (a) with at least one of methane and methanol as carbon source, wherein isoprene is produced.

18. The method of claim 17 wherein the recombinant methylotrophic cells comprise further at least one of:
a) at least one genetic modification which increases activity of at least one enzyme of the DXP pathway, other than isopentenyl diphosphate isomerase activity, wherein the activity is higher than in cells without the modification;
b) at least one genetic modification which decreases activity of at least one endogenous gene encoding an enzyme in a pathway downstream of IPP and DMAPP; and
c) at least one genetic modification which increases activity of at least one enzyme in a pathway for at least one of methane assimilation and methanol assimilation.
